(19) United States
(12) Patent Application Publication
(10) Pub. No.: US 2023/0352185 A1
(43) Pub. Date: Nov. 2, 2023

(54) Method and System for Generating a User Tunable Representation of Glucose Homeostasis in Type 1 Diabetes Based on Automated Receipt of Therapy Profile Data

(71) Applicant: University of Virginia Patent Foundation, Charlottesville, VA (US)

(72) Inventors: Boris P. KOVATCHEV, Charlottesville, VA (US); Marc D. BRETON, Charlottesville, VA (US); Ke WANG, Charlottesville, VA (US); Patricio COLMEGNA, Charlottesville, VA (US)

(21) Appl. No.: 18/041,659

(22) PCT Filed: Aug. 13, 2021
(86) PCT No.: PCT/US2021/045936

§ 371 Date: Feb. 14, 2023

Related U.S. Application Data
(60) Provisional application No. 63/065,948, filed on Aug. 14, 2020.

(51) Int. Cl. G16H 20/17; G16H 20/60; G16H 50/50

(52) U.S. Cl. CPC G16H 50/50; G16H 20/17; G16H 20/60; A61B 5/14532

(57) ABSTRACT

A method, system, and computer-readable medium are provided for modeling a time-varying representation of the glucose homeostasis of a patient with Type 1 diabetes (T1D) according to a computational model therefor. The model implements a reconstruction of data supporting a glucose time series for the patient, and based on the reconstruction, further implements model personalization and a variability control (VC) signal accounting for insulin sensitivity so as to enable the patient to learn an effect of adjustment to one or more portions of the data. Such knowledge is acquired upon a replay of the reconstruction implementing the adjustment.

### CROSS-REFERENCE TO RELATED APPLICATION

This Application is a U.S. national stage filing under 35 U.S.C. § 371 of International Application No. PCT/US2021/045936, filed Aug. 13, 2021, which claims the benefit of U.S. Provisional Application 63/065,948, filed Aug. 14, 2020, each of which is incorporated herein by reference in its entirety.

### FIELD OF THE DISCLOSURE

Disclosed embodiments relate to determination of impaired glucose homeostasis in individuals with Type 1 diabetes mellitus (T1DM; herein T1D), and more specifically, such determination as enabled by user interactive tuning of modeled representation for such homeostasis so as to forecast an effect thereon in response to the tuning, the representation being informed by the automated receipt of underlying data therefor.

### BACKGROUND

T1D is an autoimmune condition resulting in absolute insulin deficiency and a life-long need for exogenous insulin.

Benefits of intensive insulin treatment (IIT) on improving glucose control in individuals with T1D, and as a consequence, on reducing the risk for developing diabetic complications, have been known since the Diabetes Control and Complications trial (DCCT).^{1,2} For example, it has been reported that IIT reduces the risk of any cardiovascular disease event by 42%.³ However, most people with T1D still do not achieve the glycated hemoglobin (HbA1c) target recommended by the American Diabetes Association (ADA),⁴ in part due to the lack of education or self-management support.⁵ Improving self-care practices is known to be fundamental to successful treatment, because the vast majority of the day-to-day care in diabetes is handled by the patients themselves and/or their families.⁶

Advanced insulin therapy relies on key individual therapy profiles such as carbohydrate ratio (CR: grams of carbohydrate per unit of insulin), insulin sensitivity factor or correction factor (ISF or CF: mg/dL of glucose per unit of insulin), and insulin basal rate (IBR).⁷ Therapy profiles are not only used in standard open-loop basal-bolus treatments like sensor-augmented pump (SAP) therapy, but also by automated insulin dosing systems like Control-IQ® technology.⁸ Structured education programs are available to empower people with T1D to manage their disease and estimate individualized IBR, CR, and CF profiles.⁹ However, despite these efforts, achieving tight glycemic control without increasing the risk for hypoglycemia still poses a difficult, life-long, optimization problem for people with T1D.¹⁰ Periodic adjustments of IBR, CR, and CF profiles are needed based on review of self-monitoring blood glucose (SMBG) or continuous glucose monitoring (CGM) data.⁹ When a new pattern of glycemic risk is identified, patients need to tune their therapy profiles accordingly. This is a time-consuming, trial-and-error process that usually requires data to be downloaded from multiple devices, and experimentation that can take weeks to complete.

The rapid evolution of information technology has facilitated the management of chronic diseases,^{11,12} including diabetes.¹³ Among the vast number of technology-enabled applications,¹⁴ only few allow user/data interactions by means of interactive simulations.¹⁵⁻¹⁸ One example is the Karlsburg Diabetes Management System, KADIS®.¹⁶ This simulation-based decision support tool provides therapy recommendations based on an internal description of the patient’s insulin-glucose dynamics. The core metabolic model is represented by a fourth-order differential equation system that can be individualized using patient demographic information, and a structured measurement plan that requires patients to make logbook entries of self-control data, such as time and amount of meal and insulin intakes. The process needed to identify risk patterns and simulate therapy adjustments is performed by specialized operators, i.e., other than by a patient. The performance of this system as a decision support tool has been evaluated in several studies, showing promising results.¹⁶ Another example of a simulation-based application is DiasNet.^{19,20} Unlike KADIS®, this system was also meant to be used by patients as an educational tool. Despite being developed in the early 2000s, DiasNet presented a novel concept where patients were able to simulate changes to their insulin doses and meal intakes by means of an Internet application equipped with a user interface. Glucose predictions were made using a two-compartment model of glucose metabolism implemented in a casual probabilistic network. Model parameters were adjusted using diabetes data that patients needed to manually enter into the system.

Thus, it would be desirable to overcome the above disadvantages including the absence of user interaction (as with KADIS®) and a user burden arising from manual data entry to afford individuals with T1D an ability to achieve a tailored forecast of their glucose homeostasis that is based upon automation of receipt of modeling data underlying correspondingly instituted modeling therefor. In doing so, it is suggested that such individuals may obtain a more appropriate understanding of their T1D, with an assurance as to the accuracy of the data that may be processed when reaching that understanding.

### SUMMARY

It is to be understood that both the following summary and the detailed description are exemplary and explanatory and are intended to provide further explanation of the present embodiments as claimed. Neither the summary nor the description that follows is intended to define or limit the scope of the present embodiments to the particular features mentioned in the summary or in the description. Rather, the scope of the present embodiments is defined by the appended claims.

An embodiment may include a processor-implemented method for modeling a time-varying representation of the glucose homeostasis of a patient with Type 1 diabetes (T1D) according to a computational model driven by the processor, the method including retrieving from a storage a dataset for said patient comprising continuous glucose monitoring (CGM), insulin, and meal records collected from one or more devices associated with the patient, the dataset being automatedly deposited into the storage at one or more predetermined time intervals; determining, according to operation of the model on the dataset, a subset (θ^{r}) of most-impacting, low-correlated model parameters, along with a variability control (VC) signal accounting for insulin sensitivity (IS) of the patient; formulating, based on the model being informed by each of the subset θ^{r}, the VC signal and the dataset, a reconstructed glucose time series for the patient; and introducing to the model patient provided modification of one or more of the insulin and meal records. The method may further include, based on said modification, generating by the model a replay of the reconstructed glucose time series for the patient that reflects an effect of the modification.

The modification may be provided via an interface operably coupled to the processor.

The VC signal may be described as a truncated Fourier series capturing daily variation in IS.

The VC signal may be described as including a number of harmonics of the truncated Fourier series and a predetermined magnitude of a tuning parameter selected to penalize a power of the adjusted VC signal.

The VC signal may modulate an impact of insulin on endogenous glucose production and insulin-dependent glucose utilization when generating, respectively, said reconstructed glucose time series and said replay thereof.

θ^{r} may be a parameter subset of the model including at least insulin clearance (CL), distribution volume of glucose (V_{g}), a first diffusion constant of the model (k₁), basal endogenous glucose production (EGP_{b}), a second diffusion constant of the model (k₂), and liver glucose effectiveness (kₚ₂).

Respective embodiments may further include a relative system and computer-readable medium commensurate with the embodied method above.

In certain embodiments, the disclosed embodiments may include one or more of the features described herein.

### BRIEF DESCRIPTION OF THE DRAWINGS

The accompanying drawings, which are incorporated herein and form a part of the specification, illustrate exemplary embodiments and, together with the description, further serve to enable a person skilled in the pertinent art to make and use these embodiments and others that will be apparent to those skilled in the art. Embodiments herein will be more particularly described in conjunction with the following drawings wherein:

FIG. 1 illustrates a high-level schematic diagram of a system architecture of a Web-Based Simulation Tool (WST) for effecting tuning of glucose homeostasis representation, according to embodiments herein;

FIG. 2 illustrates a schematic diagram of dynamic modeling which may be implemented by the WST of FIG. 1, according to embodiments herein;

FIG. 3 illustrates, in accordance the Food and Drug Administration (FDA)-accepted University of Virginia (UVA)/Padova time-invariant model for generating a computational representation of impaired glucose homeostasis for an individual with T1D, an array of relevant model parameters as against a predetermined selection threshold therefor as gauged by a predetermined importance factor;

FIG. 4 illustrate, relative to the parameters of FIG. 3 falling within the predetermined selection threshold, pairwise comparison with respect to collinearity among given ones of such parameters, according to embodiments herein;

FIG. 5 illustrates, a fill contour plot of mean Final Prediction Error (FPE) values as derived from a validation set of one-day glucose and insulin vectors with respect to a predetermined cohort of in silico subjects, according to embodiments herein;

FIGS. 6A and 6B illustrate, with respect to a prediction accuracy of a replay engine of the WST of FIG. 1 as to basal insulin adjustment (using synthetic data), an evolution of Root Mean Squared Error (RMSE) in which vertical lines indicate +/- 1 standard deviation (SD) (FIG. 6A), and an error grid analysis (EGA) as to all generated in silico subjects with respect to 0% and 25% adjustment (FIG. 6B), assuming a sampling time of 30 minutes and according to embodiments herein;

FIGS. 7A and 7B illustrate, with respect to a prediction accuracy of the replay engine of the WST of FIG. 1 as to meal bolus adjustment (using synthetic data) among no adjustment, +30 minutes and -30 minutes, an evolution of RMSE in which vertical lines indicate +/- 1 SD (FIG. 7A), and an error grid analysis (EGA) as to all generated in silico subjects with respect to 0% and 25% adjustment (FIG. 7B), assuming a sampling time of 30 minutes and according to embodiments herein;

FIGS. 8A and 8B illustrate, with respect to glucose reconstruction and prediction as to mixed meals, a glucose rate of appearance of a selected meal (FIG. 8A), and Continuous Glucose Monitor (CGM) measurements relative to glucose predictions when (a) meal bolus at breakfast is not adjusted, (b) meal bolus at breakfast is increased 25% and delivered 30 minutes beforehand, and (c) meal bolus at breakfast is decreased 25% and delivered 30 minutes after mealtime, according to embodiments herein;

FIG. 9 illustrates a plot of average weighted residuals relative to real data underlying the modeling of the WST of FIG. 1, wherein vertical lines indicate +/- 1 SD and according to embodiments herein;

FIG. 10 illustrates a plot of glucose reconstruction for an individual providing real data underlying modeling as performed by the WST of FIG. 1, whereas glucose and a variability component signal accounting for variation in insulin sensitivity over time are analyzed, according to embodiments herein;

FIG. 11 illustrates a comparison between time responses among in silico subjects to a given experiment to actual glucose profile, according to embodiments herein;

FIGS. 12A-12C illustrate median glucose responses to adjustment in meal size and/or insulin bolus, with variation between 0% and 100% in 25% increments, according to embodiments herein;

FIG. 13 illustrates a dashboard view as may be implemented by the WST of FIG. 1 to display on a given interface a display panel effecting a reconstruction of received glucose homeostasis, as well as a replay panel effecting a result of the replay engine of the WST of FIG. 1 insofar as such engine may approximate an effect of a modification of one or more of homeostasis-impacting data on the reconstruction, according to embodiments herein;

FIG. 14 illustrates a plot of percentage time in target glucose range 70-180 mg/dl relative to baseline measurement and during use of the WST according to FIG. 1, according to embodiments herein;

FIG. 15 illustrates an exemplary monitoring of implementation of a replay engine result scenario as adopted by an individual user, whereas when glucose profile is compared to insulin basal rate (IBR), a reduction in IBR evinces decreased risk of hypoglycemia, according to embodiments herein; and

FIG. 16 is a high-level block diagram of information processing according to FIGS. 1 and 2.

### DETAILED DESCRIPTION

The present disclosure will now be described in terms of various exemplary embodiments. This specification discloses one or more embodiments that incorporate features of the present embodiments. The embodiment(s) described, and references in the specification to “one embodiment”, “an embodiment”, “an example embodiment”, etc., indicate that the embodiment(s) described may include a particular feature, structure, or characteristic. Such phrases are not necessarily referring to the same embodiment. The skilled artisan will appreciate that a particular feature, structure, or characteristic described in connection with one embodiment is not necessarily limited to that embodiment but typically has relevance and applicability to one or more other embodiments.

In the several figures, like reference numerals may be used for like elements having like functions even in different drawings. The embodiments described, and their detailed construction and elements, are merely provided to assist in a comprehensive understanding of the present embodiments. Thus, it is apparent that the present embodiments can be carried out in a variety of ways, and does not require any of the specific features described herein. Also, well-known functions or constructions are not described in detail since they would obscure the present embodiments with unnecessary detail.

The description is not to be taken in a limiting sense, but is made merely for the purpose of illustrating the general principles of the present embodiments, since the scope of the present embodiments are best defined by the appended claims.

It should also be noted that in some alternative implementations, the blocks in a flowchart, the communications in a sequence-diagram, the states in a state-diagram, etc., may occur out of the orders illustrated in the figures. That is, the illustrated orders of the blocks/communications/states are not intended to be limiting. Rather, the illustrated blocks/communications/states may be reordered into any suitable order, and some of the blocks/communications/states could occur simultaneously.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedure, Section 2111.03.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments. The word “exemplary” is used herein to mean “serving as an example, instance, or illustration.” Any embodiment described herein as “exemplary” is not necessarily to be construed as preferred or advantageous over other embodiments. Additionally, all embodiments described herein should be considered exemplary unless otherwise stated.

It should be appreciated that any of the components or modules referred to with regards to any of the embodiments discussed herein, may be integrally or separately formed with one another. Further, redundant functions or structures of the components or modules may be implemented. Moreover, the various components may be communicated locally and/or remotely with any user/clinician/patient or machine/system/computer/processor. Moreover, the various components may be in communication via wireless and/or hardwire or other desirable and available communication means, systems and hardware. Moreover, various components and modules may be substituted with other modules or components that provide similar functions.

It should be appreciated that the device and related components discussed herein may take on all shapes along the entire continual geometric spectrum of manipulation of x, y and z planes to provide and meet the anatomical, environmental, and structural demands and operational requirements. Moreover, locations and alignments of the various components may vary as desired or required.

It should be appreciated that various sizes, dimensions, contours, rigidity, shapes, flexibility and materials of any of the components or portions of components in the various embodiments discussed throughout may be varied and utilized as desired or required.

It should be appreciated that while some dimensions are provided on the aforementioned figures, the device may constitute various sizes, dimensions, contours, rigidity, shapes, flexibility and materials as it pertains to the components or portions of components of the device, and therefore may be varied and utilized as desired or required.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

Ranges may be expressed herein as from “about” or “approximately” one particular value and/or to “about” or “approximately” another particular value. When such a range is expressed, other exemplary embodiments include from the one particular value and/or to the other particular value.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

Some references, which may include various patents, patent applications, and publications, are cited in a reference list and discussed in the disclosure provided herein. The citation and/or discussion of such references is provided merely to clarify the description of the present disclosure and is not an admission that any such reference is “prior art” to any aspects of the present disclosure described herein. In terms of notation, “[n]” corresponds to the n^{th} reference in the list. All references cited and discussed in this specification are incorporated herein by reference in their entireties and to the same extent as if each reference was individually incorporated by reference.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, 4.24, and 5). Similarly, numerical ranges recited herein by endpoints include subranges subsumed within that range (e.g. 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

Referring to FIG. 1, there is shown a high-level schematic diagram of a system architecture of a Web-Based Simulation Tool (WST) 20 (as shown to encompass one or more components and processes effected thereby as provided in the greyed region) for effecting tuning of glucose homeostasis representation. In this regard, it is to be understood that, in addition to or in connection with, the WST 20 may be equipped with all appropriate hardware and/or software necessary for implementing operations as are discussed herein. In a larger scope, the WST 20 may be a constituent component of a glucose analyzer 21 implementing each of data collection A, model optimization B, and user interaction C.

In other words, the glucose analyzer 21 may include, with respect to data collection A and for example, a Tandem t:slim X2™ insulin pump 22 in communication with a constituent smart device 24 configured to implement Tandem’s t:connect® web application for enabling viewing and management of information including glucose level, insulin boluses, and insulin on board (IOB). The insulin pump 22 may be cooperable with a continuous glucose monitor (CGM) 23, such that an artificial pancreas (AP) may be provided. In operation, data uploads from a given insulin pump 22 and CGM 23 to a Tandem cloud server 26 occur approximately every two (2) hours or in response to a qualifying event. Based upon interaction with the t:connect® web application, WST 20 is enabled to fetch, as at 28, the insulin pump and CGM data, and interpret and save, as at 30, embodied JavaScript Object Notation (JSON) data.

As may be understood, the JSON files may include a dataset defining glucose, insulin, meal, and therapy profile (IBR, CR, and CF) records (hereinafter “dataset”) discernable by the WST 20 and processed such that (i) samples are sorted by ascending time; (ii) timestamps are synchronized; (iii) duplicate records are removed; (iv) gaps are located and their extension measured; (v) qualifying events are detected (extended boluses, declined corrections, temporary basal rates, and overwritten doses); (vi) gaps are filled depending on the type of data (for instance, linear interpolation for CGM data, and previous values for IBR); and (vii) days of data are classified as either playable (a model can be obtained) or non-playable according to a data quality assessment (DQA) checklist. The DQA includes glucose and insulin gaps no longer than two (2) hours during the day and three (3) hours during the night (12:00 AM to 06:00 AM), and the existence of IBR, CR, and CF profiles.

When processing the samples, the WST 20 may implement a meal detection algorithm. In particular, and after cleaning the data, the meal detection algorithm is run to adjust the time of announced meals, and detect potential unannounced meals and hypo-treatments. Given a one-day-long data set, the algorithm: (i) captures the mealtimes of reported meals; (ii) computes the product between the first and second derivatives of the glucose signal (µ), both estimated using a Savitzky-Golay filter of polynomial order three (3) and frame length 13; (iii) adjusts the mealtime of each reported meal by looking for a peak in µ around the informed time and moving the mealtime to the base of the potential peak; (iv) finds all the peaks in µ that meet certain criteria with respect to height (0.0075 mg²/dL²/min³), prominence (0.0025 mg²/dL²/min³), and separation (30 min); (v) defines potential mealtimes at the peaks’ bases (fixed size based on historical records); (vi) classifies an event as a hypo-treatment based on a glucose threshold and the size of the peak; (vii) if the event is not recognized as a hypo-treatment, then the algorithm evaluates the glucose deviation to determine if it can be classified as a meal; and (viii) appends the vector of detected, unannounced meals to the vector of adjusted, informed meals, eliminating repetitions.

Subsequently, the WST 20 proceeds as at B to undertake model optimization with respect to received glucose, insulin, and meal data for a given user of the insulin pump 22. In doing so, the WST 20 may initiate data processing for scheduling of jobs as at 32 in preparation of a virtual image of the user with T1D, whereas such image may be used for purposes of tuning of glucose homeostasis representation as to that user. In this regard, the term virtual image may be understood as a high-precision computational representation of the impaired glucose homeostasis as relates to T1D.

As discussed in more detail below, each of the data processing 32 and implementation of the virtual image generator 34 may enact the Food and Drug Administration (FDA)-accepted University of Virginia (UVA)/Padova time-invariant model (UVA model) for generating a computational representation of impaired glucose homeostasis for an individual with T1D.³⁵ In particular, the generator 34 may coordinate with a high performance computing service 35 to achieve generation of the virtual image. Based on this, the UVA model may afford each of a “reconstruction” of an originally received glucose time series, and thereafter, in response to selective user modification for the series, a “replay” of the reconstructed time series so as to approximate an effect of the modification, i.e., a modified/alternate treatment strategy. Creation, access, and storage of the data processing 32 and virtual image generation 34 results may be implemented using, optionally, a relational database service (RDS) 36 as enabled by, optionally, Amazon Web Services (AWS®) or other provider of like services and platforms.

In use, the WST 20 may be configured to attain an ability for user interaction C via implementation of user interface 39 enabling user visualization of a display panel 38 and a replay panel 40 as regards the aforementioned operations of the UVA model. Instances of the replay panel, as will be understood, may be triggered in response to an HTTP request initiated by the user.

More specifically, the first page that a user may look at is the Login page, where he/she can gain access to WST 20 by entering his or her e-mail address and password, as relates to mail server 37. Once logged in, the user is redirected to a Dashboard page that is the main screen of the application presented by the WST 20, where the user may visualize his/her data and run simulations regarding the above-discussed tuning of homeostasis representation. WST 20 is also equipped with (i) a Support page where the user can review frequent questions, watch tutorial videos, and run an interactive example that guides them through learning the WST 20 system; (ii) a Contact page where users can request more information about system functionalities; and (iii) a Profile page where users can find basic information about their accounts.

In the Display panel 38, the user may select, with respect to processing according to the UVA model, a particular date range using a calendar. The user may study a single day or combine multiple days for more systematic reviews. The user may also control the amount of information displayed on the screen, showing or hiding extra indices like glucose variability, and risks for hypo- and hyperglycemia.

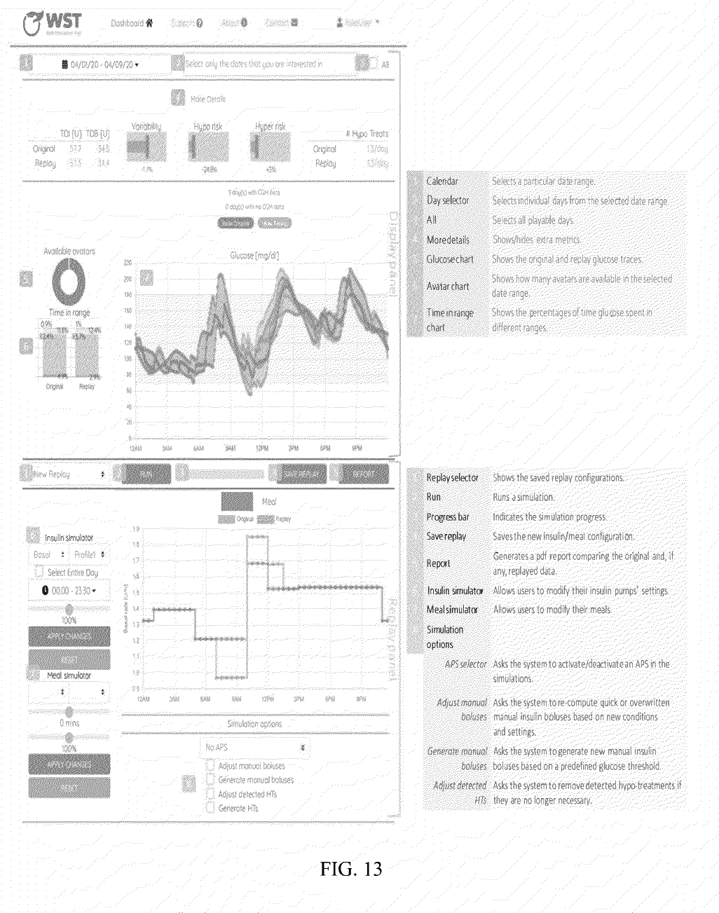

In the Replay panel 40, the user may select varying pump parameters, like IBR, CR, and CF profiles, a particular time interval, and then modulate the signal amplitude by just moving a slider. A similar procedure may be executed with respect to meals, such that the user may alter the time and size of all meals at the same time, or on a one-by-one basis. To perform a simulation enabling the aforementioned replay, users just need to tap a Run button (see FIG. 13). This will make the system carry out a replay simulation, which status is monitored by a progress bar. Results of the replay simulation are shown as against the reconstructed data to facilitate comparison therebetween. Once the simulation is done, users may generate a report highlighting all made changes, and save their new insulin/meal configuration - WST 20 may store replay configurations temporarily, allowing users to easily compare up to three different replays.

Internal to the user interactions C, the WST 20 may further store and format logs containing records of user activity and events, such that the same may later be analyzed in person by a clinical research director in order to assist the user with additional refinement of his/her T1D treatment strategy.

Discussed below is the general framework of modeling enabling the user to achieve the aforementioned ability to tune his/her homeostasis representation, as implemented by the UVA model.

Precision medicine, or the adaptation of standard, population-based treatments to the individual needs of a specific patient, necessitates the representation of the underlying dynamics that link treatment to clinical outcome, and in the case of diabetes, the mapping between the data generated by the patient under standard of care and a mathematical representation of glucose metabolism. Intuitively, this concept combines three key elements: right treatment, right time, and right patient. Yet, when endeavoring to achieve this representation exists insofar as real-life CGM traces exhibit a variability that cannot be fully recreated in simulation, particularly because certain behavioral influences are difficult to model. To overcome this limitation, we define a net-effect signal as an (additive) input that best explains the correlated time series of CGM values, and insulin pump data, accounting for a linear population-level model used to describe the patient’s physiology. In order to cover this inherent uncertainty, in Ramkisson et al.,³³ an Unscented Kalman Filter (UKF) is used to estimate a disturbance signal that represents all unmodeled glucose variations. In Goodwin et al.,³⁴ the authors propose the Metabolic Digital Twin Envelope (MDTE), an envelope of low-order-model responses that capture the impact of subject-specific metabolic variability (uncertainty).

More particularly, we implement herein a time-varying virtual image of a patient with T1D for replay purposes. In this context, the term virtual image is used as a high-precision computational representation of the impaired glucose homeostasis in T1D for a particular individual. Given a dataset containing daily CGM, meal and insulin pump records, the time-invariant version of the UVA model is individualized, including determination of a variability component (VC) signal ω(t) that captures widely accepted daily variations in insulin sensitivity (IS).²⁹ Based on the dataset, the UVA model identifies a subset θ^{r}of most-impacting, low-correlated model parameters supporting glucose level assessment. Based on this subset θ^{r}, the UVA model may then generate ω(t), whereby θ^{r} and ω(t) may then be fed back into the model to reproduce the original glucose time series (Reconstruction), and approximate the effect of a modified treatment strategy (Replay), as discussed below.

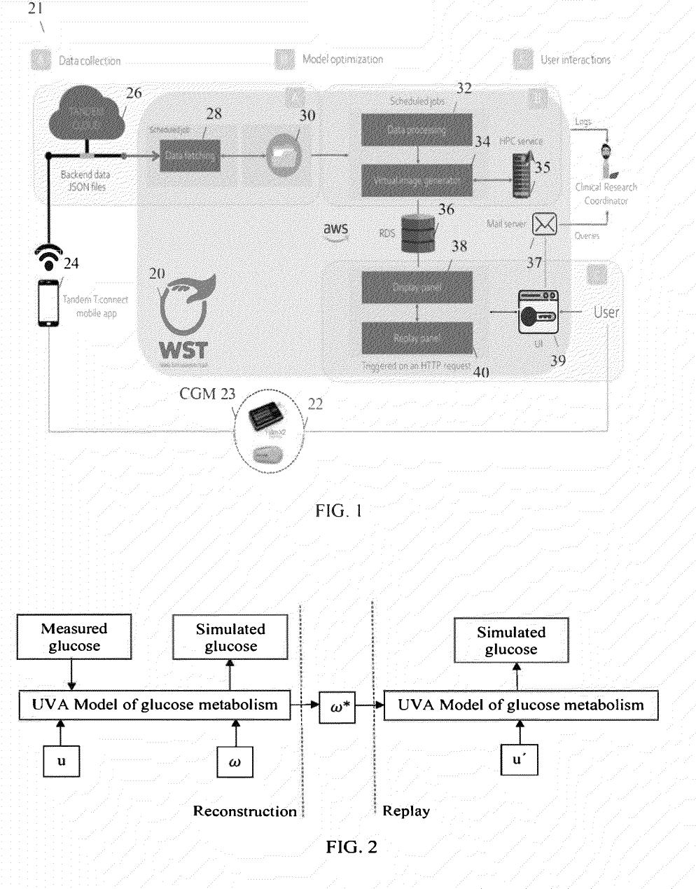

In referring to FIG. 2, there is illustrated the framework of dynamic modeling as performed by the WST when attaining reconstruction and replay as to meal and insulin data of a user. Therein, measured glucose and original meal and insulin inputs u are fed into the UVA model to identify θ^{r}, and then generate VC signal ω, whereupon a simulated glucose time series may be generated to obtain the aforementioned reconstruction. Once reconstruction is obtained, user driven modifications thereto may be provided in the form of adjusted meal and/or insulin inputs u′ to enable the user to evaluate, based on the identification of θ^{r} and the estimated VC signal ω*, a replay of the reconstruction. This way, the user may be enabled to assess an effect of those modifications in regard to a change in his/her glucose level.

### Method

The proposed procedure consists in identifying the most sensitive parameter subset θ^{r} of the UVA model along with a VC signal ω* based on subject-specific glucose, insulin, and meal data. All 100 parameter vectors of the in silico adult cohort of the UVA/Padova simulator are used as initial conditions of the optimization problem.

Since the UVA model is high-dimensional, the first step was to determine the most suitable subset of model parameters to be identified. This not only helps reduce overfitting, but also the computational load. To this end, given the full vector of model parameters θ, a subset θ^{r} was determined based on the global ranking of parameters and collinearity analysis. This procedure was implemented in the Matlab® toolbox AMIGO2, and migrated into the UVA’s High-Performance Computing (HPC) system (https://www.rc.virginia.edu).

Thirty-eight sets of CGM, insulin, and meal (including hypo-treatments) vectors with a sampling time of 5 min, and duration of 20 to 24 hours were extracted from data collected during 48-hour supervised hotel admissions of a previous clinical trial. Fifteen patients with T1D (eleven women and four men) were included in this analysis. The mean ± standard deviation (SD) age was 41±11 years, glycated hemoglobin (HbA1c) was 7.41±0.97%, body mass index (BMI) was 28.33±4.55 kg/m², duration of diabetes was 25±10 years, and total daily insulin (TDI) was 48.88±17.39 units (U). For each day, the timeline of the protocol included breakfast (8:00-9:00), 45-min exercise activity (~11:00), lunch (12:00-13:00) and dinner (18:00-19:00). This trial was designed to include combinations of challenging meals (e.g., large and fatty meals) and exercise bouts to evaluate the UVA decision support systems (DSS) under conditions of large glucose variability.

### The UVA Model

Presented below is the representation of the UVA model, together with variables/fluxes explained in Table 1 and parameters explained in Table 2.

### Glucose Subsystem

$$\begin{matrix}{\dot{G}}_{p} \left(t\right) = E G P \left(t\right) + R a \left(t\right) - {U}_{i i} \left(t\right) - E \left(t\right) - \\ {k}_{1} {G}_{p} \left(t\right) + {k}_{2} {G}_{t} \left(t\right)\end{matrix} \qquad \text{(1)}$$

$${\dot{G}}_{t} \left(t\right) = - {U}_{i d} \left(t\right) + {k}_{1} {G}_{p} \left(t\right) - {k}_{2} {G}_{t} \left(t\right) \qquad \text{(2)}$$

$$G \left(t\right) = \frac{{G}_{p} \left(t\right)}{{V}_{g}} \qquad \text{(3)}$$

$${G}_{s} \left(t\right) = - {k}_{s c} \left[{G}_{s} \left(t\right) - {G}_{p} \left(t\right)\right] \qquad \text{(4)}$$

$$E G P \left(t\right) = {k}_{p 1} - {k}_{p 2} {G}_{p} \left(t\right) - {k}_{p 3} {I}_{d} \left(t\right) + ψ {X}^{H} \left(t\right) \qquad \text{(5)}$$

$${k}_{p 1} = E G {P}_{b} + {k}_{p 2} {G}_{b} {V}_{g} + {k}_{p 3} {I}_{b} \qquad \text{(6)}$$

$${U}_{i i} \left(t\right) = {F}_{c n s} \qquad \text{(7)}$$

$${U}_{i d} \left(t\right) = \frac{\left[{V}_{m 0} + {V}_{m x} X \left(t\right) \left(1 + {r}_{3} ⋅ r i s k\right)\right] {G}_{t} \left(t\right)}{{K}_{m 0} + {G}_{t} \left(t\right)} \qquad \text{(8)}$$

$$r i s k = \left\{\begin{matrix}0 & \text{(9)} G \geq {G}_{b} \\ 10 {\left[f \left(G\right)\right]}^{2} & \text{if} 60 \leq G < {G}_{b} \\ 10 {\left[f \left(60\right)\right]}^{2} & \text{if} G < 60\end{matrix}\right)$$

$$f \left(G\right) = l o g {\left(G\right)}^{{r}_{1}} - l o g {\left({G}_{b}\right)}^{{r}_{1}} \qquad \text{(10)}$$

$$E \left(t\right) = \left\{\begin{matrix}{k}_{e 1} \left[{G}_{p} \left(t\right) - {k}_{e 2}\right] & {G}_{p} \left(t\right) > {k}_{e 2} \\ 0 & {G}_{p} \left(t\right) \leq {k}_{e 2}\end{matrix}\right) \qquad \text{(11)}$$

$${\dot{X}}^{H} \left(t\right) = - {k}_{H} {X}^{H} \left(t\right) + {k}_{H} max \left[\left(H \left(t\right) - {H}_{b}\right) , 0\right] \qquad \text{(12)}$$

### Subcutaneous Insulin Delivery Subsystem

$${\dot{I}}_{s c 1} = - \left({k}_{d} + {k}_{a 1}\right) {I}_{s c 1} \left(t\right) + u \left(t - τ\right) \qquad \text{(13)}$$

$${\dot{I}}_{s c 2} = {k}_{d} {I}_{s c 1} \left(t\right) - {k}_{a 2} {I}_{s c 2} \left(t\right) \qquad \text{(14)}$$

$$R {a}_{I s c} \left(t\right) = {k}_{a 1} {I}_{s c 1} \left(t\right) + {k}_{a 2} {I}_{s c 2} \left(t\right) \qquad \text{(15)}$$

### Insulin Subsystem

$${\dot{I}}_{l} \left(t\right) = - \left({m}_{1} + {m}_{3}\right) {I}_{l} \left(t\right) + {m}_{2} {I}_{p} \left(t\right) \qquad \text{(16)}$$

$${\dot{I}}_{p} \left(t\right) = - \left({m}_{2} + {m}_{4}\right) {I}_{p} \left(t\right) + {m}_{1} {I}_{l} \left(t\right) + R {a}_{I s c} \left(t\right) \qquad \text{(17)}$$

$$I \left(t\right) = \frac{{I}_{p} \left(t\right)}{{V}_{i}} , {m}_{2} = \frac{3 C L}{5 H {E}_{b} {V}_{i} B W} \qquad \text{(18)}$$

$${m}_{3} = \frac{H {E}_{b} {m}_{1}}{1 - H {E}_{b}} , {m}_{4} = \frac{2 C L}{5 {V}_{i} B W} \qquad \text{(19)}$$

$${\dot{I}}_{1} \left(t\right) = - {k}_{i} \left[{I}_{1} \left(t\right) - I \left(t\right)\right] \qquad \text{(20)}$$

$${\dot{I}}_{d} \left(t\right) = - {k}_{i} \left[{I}_{d} \left(t\right) - {I}_{1} \left(t\right)\right] \qquad \text{(21)}$$

$${V}_{m 0} = \frac{\left(E G {P}_{b} - {F}_{c n s}\right) \left({K}_{m 0} + {G}_{t b}\right)}{{G}_{t b}} \qquad \text{(22)}$$

$${G}_{t b} = \frac{{F}_{c n s} - E G {P}_{b} + {k}_{1} {G}_{p b}}{{k}_{2}} \qquad \text{(23)}$$

$$\dot{X} \left(t\right) = - {p}_{2 U} X \left(t\right) + {p}_{2 U} \left[I \left(t\right) - {I}_{b}\right] \qquad \text{(24)}$$

### Glucagon Kinetics

$$\dot{H} \left(t\right) = - \frac{n}{{V}_{g n}} H \left(t\right) + S {R}_{H}^{s} \left(t\right) + S {R}_{H}^{d} \left(t\right) \qquad \text{(25)}$$

$$\dot{S} {R}_{H}^{s} \left(t\right) = \left\{\begin{matrix}- ρ \left[S {R}_{H}^{s} \left(t\right) - S {R}_{H}^{b}\right] \text{if} G \left(t\right) \geq {G}_{t h} \\ - ρ \left[S {R}_{H}^{s} \left(t\right) - \text{Γ}\right] \text{if} G \left(t\right) < {G}_{t h}\end{matrix}\right) \qquad \text{(26)}$$

$$\text{Γ} = max \left(\frac{σ \left[{G}_{t h} - G \left(t\right)\right]}{I \left(t\right) - {I}_{t h} + 1} + S {R}_{H}^{b} , 0\right) \qquad \text{(27)}$$

$$S {R}_{H}^{d} \left(t\right) = δ ⋅ max \left(- \frac{d G \left(t\right)}{d t} , 0\right) \qquad \text{(28)}$$

### Oral Glucose Absorption Subsystem

$${Q}_{s t o} \left(t\right) = {Q}_{s t o 1} \left(t\right) + {Q}_{s t o 2} \left(t\right) \qquad \text{(29)}$$

$${\dot{Q}}_{s t o 1} \left(t\right) = - {k}_{m a x} {Q}_{s t o 1} \left(t\right) + m \left(t\right) \qquad \text{(30)}$$

$${\dot{Q}}_{s t o 2} \left(t\right) = - {k}_{e m p t} \left({Q}_{s t o}\right) {Q}_{s t o 2} \left(t\right) + {k}_{m a x} {Q}_{s t o 1} \left(t\right) \qquad \text{(31)}$$

$$\begin{matrix}{\dot{Q}}_{g u t} \left(t\right) = - {k}_{a b s} {Q}_{g u t} \left(t\right) + {k}_{e m p t} \left({Q}_{s t o}\right) {Q}_{s t o 2} \left(t\right) \\ {k}_{e m p t} = {k}_{m i n} + \frac{{k}_{m a x} - {k}_{m i n}}{2} \left\{tanh \left[α \left({Q}_{s t o} - b D\right)\right]\right\}\end{matrix} \qquad \text{(32)}$$

$$- tanh \left[β \left({Q}_{s t o} - d D\right)\right] \left(+ 2\right\} \qquad \text{(33)}$$

$$α = \frac{5}{2 D \left(1 - b\right)} , β = \frac{5}{2 D d} \qquad \text{(34)}$$

$$R a \left(t\right) = \frac{f {k}_{a b s} {Q}_{g u t}}{B W} \qquad \text{(35)}$$

TABLE 1

| | | | |
|---|---|---|---|
| Variable | Description | Variable | Description |
| Gₚ | Glucose mass in plasma and rapidly equilibrating tissues. | Gₜ | Glucose mass in slowly equilibrating tissues. |
| G | Plasma glucose concentration. | Gₛ | Glucose mass in subcutaneous space. |
| EGP | Endogenous glucose production. | Ra | Glucose rate of appearance in plasma. |
| E | Renal excretion. | Uᵢᵢ | Insulin independent glucose utilization. |
| U_{id} | Insulin dependent glucose utilization. | F_{cns} | Glucose uptake by the brain and erythrocytes. |
| Iₚ | Insulin mass in plasma. | I_{ℓ} | Insulin mass in liver. |
| I | Plasma insulin concentration. | HE | Hepatic extraction of insulin. |
| I_{d} | Delayed insulin signal. | I₁ | Insulin signal associated with I_{d}. |
| I_{sc1} | Insulin in a non-monomeric state. | I_{sc2} | Insulin in a monomeric state. |
| m | Meal signal. | u | Insulin infusion rate. |
| Ra_{Isc} | Rate of appearance of insulin in plasma. | X | Insulin signal. |
| Oₛₜₒ | Total amount of glucose in the stomach. | Qₛₜₒ₁ | Amount of glucose in the stomach (solid phase). |
| Qₛₜₒ₂ | Amount of glucose in the stomach (triturated phase). | Q_{gut} | Amount of glucose in the intestine. |
| D | Ingested meal dose. | H | Plasma glucagon concentration. |
| SR_{H} | Glucagon secretion. | X^{H} | Delayed glucagon action on glucose production. |

TABLE 2

| | | | |
|---|---|---|---|
| Parameter | Description | Parameter | Description |
| kₚ₁ | Extrapolated EGP at zero glucose and insulin. | kₚ₂ | Liver glucose effectiveness. |
| kₚ₃ | Parameter governing amplitude of insulin action on the liver. | kₑ₁ | Glomerular filtration rate. |
| kₑ₂ | Renal threshold of glucose. | Vₘₓ | Parameter from the Michaelis Menten equation. |
| Vₘ₀ | Parameter from the Michaelis Menten equation. | Kₘ₀ | Parameter from the Michaelis Menten equation. |
| p_{2U} | Rate constant of insulin action on the peripheral glucose utilization. | k₁ | Diffusion constant of glucose subsystem. |
| k₂ | Diffusion constant of glucose subsystem. | k_{sc} | Diffusion constant of subcutaneous glucose subsystem. |
| V_{g} | Distribution volume of glucose. | Vᵢ | Distribution volume of insulin. |
| m₁ | Rate parameter of insulin subsystem. | m₂ | Rate parameter of insulin subsystem. |
| m₃ | Rate parameter of insulin subsystem. | m₄ | Rate parameter of insulin subsystem. |
| CL | Insulin clearance. | kᵢ | Rate parameter accounting for delay between insulin signal and insulin action. |
| τ | Time delay in the appearance of insulin in the first compartment after sc injection. | k_{d} | Rate parameter accounting for subcutaneous insulin kinetics. |
| kₐ₁ | Rate parameter accounting for subcutaneous insulin kinetics. | kₐ₂ | Rate parameters accounting for subcutaneous insulin kinetics. |
| k_{abs} | Rate of intestinal absorption. | kₑₘₚₜ | Rate of gastric emptying. |
| kₘₐₓ | Maximum rate of gastric emptying. | kₘᵢₙ | Minimum rate of gastric emptying. |
| b | Gastric emptying parameter. | d | Gastric emptying parameter. |
| α | Gastric emptying rate. | β | Gastric emptying rate. |
| f | Fraction of the intestinal absorption which appears in plasma. | BW | Body weight. |
| V_{gn} | Distribution volume of glucagon. | 1/ρ | Delay between static glucagon secretion and plasma glucose. |
| n | Glucagon clearance rate. | Gₜₕ | Glucose threshold controlling glucagon secretion. |
| Iₜₕ | Insulin threshold controlling glucagon secretion. | σ | Alpha-cell responsivity to glucose and insulin levels. |
| I_{b} | Basal plasma insulin concentration. | G_{b} | Basal plasma glucose concentration. |
| δ | Alpha-cell responsivity to glucose rate of change. | Ψ | Liver responsivity to glucagon. |
| k_{H} | Delay between glucagon concentration and action. | r₁ | Parameter of glucose risk function. |
| r₃ | Parameter of glucose risk function. | EGP_{b} | Basal endogenous glucose production. |
| H_{b} | Basal plasma glucagon concentration. | | |

### Ranking and Selection of Model Parameters

In the UVA model, the glucose-insulin-glucagon system is represented by a nonlinear model of thirty-five independent subject-specific model parameters θ = (θ₁, ..., θ₃₅)^{T}, with two inputs: subcutaneous insulin delivery u and meals m, and one output: subcutaneous glucose concentration y = Gₛ. To assess the individual local parameter influence, given an experiment e (one-day insulin and meal profiles), and an observable o (one-day glucose trace), relative sensitivities can be computed as:

$${s}_{p}^{e} \left({t}_{s}\right) = \frac{Δ {θ}_{p}}{Δ {G}_{s}^{e}} \frac{\partial {G}_{s}^{e}}{\partial {θ}_{p}} \left({t}_{s}\right)$$

where tₛ is the s^{th} sampling time, Δθₚ is an a priori variation range of parameter θₚ, and

$$Δ {G}_{s}^{e}$$

is an output scale factor that can be set to unity in this case, because there is only one observable. Due to the fact that parameter values are unknown a priori, one solution is to compute relative sensitivities for a sufficiently large number of parameter vectors in the feasible parameter space. The parameter space was explored using the Latin Hypercube Sampling (LHS) method, which is computationally less demanding than a Monte Carlo approach. Then, in order to quantify how sensitive a model is to a given parameter θₚ, the importance factor

$${δ}_{p}^{m s q r}$$

was computed for each experiment e:

$${δ}_{p , {θ}_{0}}^{m s q r} = \frac{1}{{n}_{l} {n}_{s}} \sqrt{{\sum }_{l = 1}^{{n}_{l}} {\sum }_{s = 1}^{{n}_{s}} {\left({\left[{s}_{p , {θ}_{0}}^{e} \left({t}_{s}\right)\right]}_{l}\right)}^{2}}$$

considering 100 different initial conditions

$${θ}_{\text{0}}^{\text{i}} , i = 1 … 100$$

of the parameter vector θ, each one linked to a particular in silico adult of the simulator. In addition, for each

$${θ}_{\text{0}}^{\text{i}} , {n}_{l} = 20$$

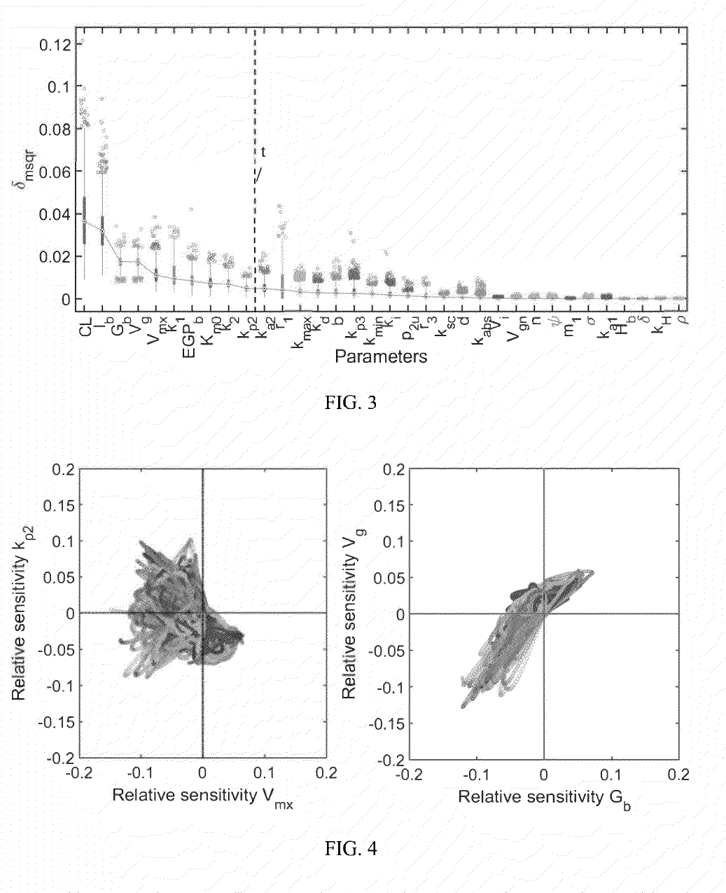

samples were generated by means of the LHS method, considering 20% deviation from nominal values. The parameter importance ranking is presented in FIG. 3, where parameters are ordered in decreasing order according to the median value of

$${δ}_{p}^{m s q r} .$$

The glucose concentration is almost insensitive to parameters related to the glucagon subsystem. At this step, the top 10 parameters that achieve a predetermined selection threshold t that explains around 85% of the total value of

$${δ}_{p}^{m s q r}$$

were selected, i.e., θ^{gr} = (CL,I_{b},G_{b},V_{g},Vₘₓ,k₁,EGP_{b},Kₘ₀,k₂,kₚ₂)^{T}, such that parameters affecting the glucagon subsystem were exempted from the selection given observed insensitivity to glucose concentration.

### Collinearity

In collinearity analysis, the joint influence of the parameters on the model output is analyzed. For example, if two columns of the sensitivity matrix are nearly dependent, then a change in the model output caused by a change in one parameter can be compensated by an appropriate change in the other parameter. This affects identifiability even if the model output is very sensitive to changes in the individual parameters. In order to drop near-collinear parameters, the following collinearity index was calculated for each experiment e:

$${γ}_{{θ}_{0}^{g r}}^{e} = \frac{1}{{min}_{\left‖β\right‖ = 1} \left‖{\overline{\text{S}}}_{{θ}_{0}^{g r \left({t}_{s}\right) β}}^{e}\right‖} = \frac{1}{\sqrt{{λ}_{{θ}_{0}^{g r}}^{e}}}$$

where

$${\overline{\text{S}}}_{{θ}_{0}^{g r}}^{e}$$

is the normalized submatrix of the sensitivity matrix associated with a given realization θ^{gr}₀ of θ^{gr}, and

$$\sqrt{{λ}_{{θ}_{0}^{g r}}^{e}}$$

is its smallest singular value. Considering a threshold for the collinearity index of 20,³² a certain degree of collinearity was detected between I_{b} and G_{b}, and G_{b} and V_{g} (see FIG. 4). Therefore, I_{b} and G_{b} were dropped, leading to a final set of eight (8) parameters θ^{r} = (CL,V_{g},Vₘₓ,k₁,EGP_{b},Kₘ₀,k₂,kₚ₂)^{T}. All dropped parameters are fixed to population values, excepting G_{b} and I_{b}, which are experiment-dependent. In this regard, G_{b} is defined as the mean glucose value during the night period, and I_{b}, as the insulin concentration obtained with the average basal insulin infusion.

### Representation of the VC Signal Ω

To include the VC signal in the UVA model, its equations 20 and 24 were modified as follows:

$${\dot{I}}_{1} \left(t\right) = - {k}_{i} \left[{I}_{1} \left(t\right) - {I}_{V C} \left(t\right)\right]$$

$$\dot{X} \left(t\right) = - {p}_{2 u} X \left(t\right) + {p}_{2 u} \left[{I}_{V C} \left(t\right) - {I}_{b}\right]$$

where I_{vc}(t) = ω(t)I(t), with ω(t) being the VC signal. Note that i₁(t) indirectly modulates parameter kₚ₃ that weights the impact of insulin on EGP, while ẋ(t) impacts on U_{id} (insulin-dependent glucose utilization). Notably, ω(t) does not alter the insulin concentration, and therefore maintains the interpretability of the model; in essence it modulates glucose absorption by modulating the model’s gain based on well-known circadian variations.²⁹

A common approach to represent a signal in finite-dimensional space is to discretize it into equal intervals along time axis. This method leads to a concise matrix expression of the corresponding linear ordinary differential equations (ODEs). However, such a method has two disadvantages: (i) computation cost is high due to the large number of variables, and (ii) there is no direct way to control the stability of the numerical process. Thus, another representation methodology is applied herein, where the VC signal is approximated in a finite function space. To this purpose, ω(t) is expressed as a truncated Fourier series:

$$ω \left(t\right) = {ϕ}_{1} + {\sum }_{i = 1}^{h} \left[{ϕ}_{2 i} cos \left(h {w}_{0} t\right) + {ϕ}_{2 i + 1} sin \left(h {w}_{0} t\right)\right]$$

where h is the number of harmonics, ϕ = (ϕ₁, ..., (ϕ₂ₕ₊₁)^{T} is the design variable vector, and ω₀ is the fundamental frequency that is set to

$$\frac{2 π}{1440}$$

1/min, i.e., one-day period. Parameter h acts as a regularization term as follows. A large value of h increases the bandwidth of the VC, but may include model variance. On the other hand, a small value of h reduces computation complexity and makes the numerical process more stable, but may increase the bias of the model.

### Optimization

For each experiment-specific glucose y = (y₁, ..., yₙ)^{T}, insulin u = (u₁, ..., uₙ)^{T}, and meal m = (m₁, ...,mₙ)^{T} vectors, the following optimization problem is formulated:

$${κ}^{∗} : = {}_{κ} f \left(κ\right)$$

with

$$κ = \left(\begin{matrix}ζ \\ ϕ\end{matrix}\right) = \left(\begin{matrix}l n \left({θ}^{r}\right) \\ ϕ\end{matrix}\right) ∈ {ℝ}^{2 ⋅ h + 9}$$

and cost function:

$$\begin{matrix}f \left(κ\right) = {\left(\text{y} - {\text{y}}_{\text{p}}\right)}^{T} {Σ}_{\text{v}} {}^{- 1} \left(\text{y} - {\text{y}}_{\text{p}}\right) + … \\ {\left(ζ - {μ}_{ζ}\right)}^{T} {Σ}_{ζ} {}^{- 1} \left(ζ - {μ}_{ζ}\right) + {\left(ϕ - {μ}_{ϕ}\right)}^{T} {Σ}_{ϕ} {}^{- 1} \left(ϕ - {μ}_{ϕ}\right)\end{matrix}$$

subject to

$${\text{lb}}_{ζ} \leq ζ \leq {\text{ub}}_{ζ}$$

$$0.5 \leq {ϕ}_{1} \leq 1.5$$

$$ω \left(t\right) \geq 0.2$$

where yₚ = (yₚ₁ ... yₚₙ)^{T} is the vector of glucose predictions,

$${Σ}_{\text{v}} = \frac{1}{64} \text{diag} \left({\text{yy}}^{T}\right)$$

represents the covariance matrix of measurement errors that, after calibration, are assumed to be independent and Gaussian with zero mean and constant coefficient of variation (CV) of 12.5% for a mean absolute relative difference (MARD) of 10%, µζ ∈ ℝ⁸ and Σ_{ζ} ∈ ℝ^{8×8} are the mean vector and covariance matrix of model parameters in logarithmic form obtained from the simulator distribution, and µ_{ϕ} =

$${\left(1 , 0 , … , 0\right)}^{T} ∈ {ℝ}^{2 ⋅ h + 1}$$

and

$${Σ}_{ϕ} ∈ {ℝ}^{\left(2 ⋅ h + 1\right) \times \left(2 ⋅ h + 1\right)}$$

with

$${Σ}_{ϕ} = \text{diag} \left(\frac{1}{γ} {\left(1 , 2 , … , 2\right)}^{T}\right)$$

are the vector and matrix used to penalize, by means of a tuning parameter γ, the power of how much the VC deviates from unity, i.e., ω(t) = 1. Finally,

$${\text{lb}}_{ζ} = {\left(l {b}_{1} , … , l {b}_{8}\right)}^{T} = {min}_{1 \leq i \leq 100} \left({T}_{i j}\right)$$

$${\text{ub}}_{ζ} = {\left(u {b}_{1} , … , u {b}_{8}\right)}^{T} = {max}_{1 \leq i \leq 100} \left({T}_{i j}\right)$$

where T = (ζ_{#1}, ..., ζ_{#100})^{T} ∈ ℝ^{100×8}, with ζ_{#i} being the design parameter vector ζ ∈ ℝ⁸ evaluated at adult #i of the simulator, and Tᵢⱼ refers to the i^{th} row and j^{th} column of T. The average value of ω(t), i.e., ϕ₁, is constrained to be between 0.5 and 1.5 to compensate potential errors in the operation point, but limiting how much the solution deviates from the nominal case ω(t) = 1). The lower bound of 0.2 is included to guarantee positive solutions, and limit the impact of erroneous data, e.g., a misreported meal, on the VC signal.

### VC Signal Ω*(t) as Expressed by Harmonics H and Tuning Parameter Γ

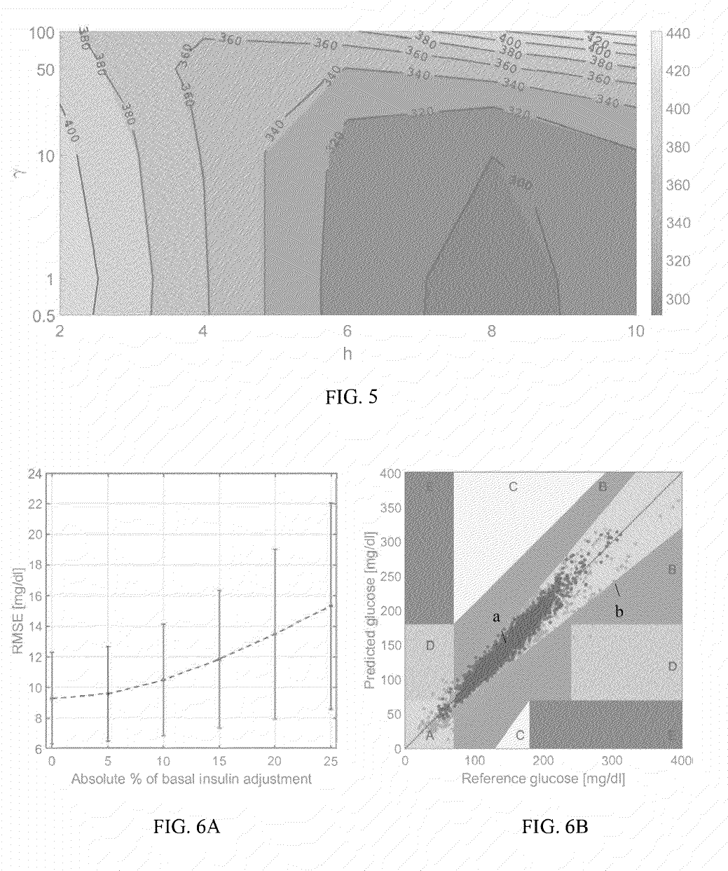

Since it is impossible to recreate exact same conditions in a real-life scenario (as existed with respect to the 38 sets of received data) to evaluate the performance of this methodology under changes to insulin and meal inputs, it is necessary to determine a (sub)-optimal combination of h and γ and thus the following in silico grid-search procedure was performed:
New fifty in silico subjects were sampled from the distribution of the UVA model.
For each new subject, two sets of one-day glucose and insulin vectors were generated: one set for training, and the other one for validation. Each set included three meals per day, CGM noise and calibrations, and a moderate 30-min exercise bout to add an extra unmodeled phenomenon. Time-varying conditions in IS were simulated as described in Visentin et al. (2018).³⁰ In this way, each in silico subject was associated with a specific daily pattern representing the level of IS (low or high) at breakfast, lunch, and dinner. Additive noise was added to nominal patterns, and step variations were smoothed using a low-pass filter. In addition to circadian variations in IS, the physiology of the dawn phenomenon was also incorporated into these simulations. The systematic rise in blood glucose concentration similarly observed in Mallard et al. (2015)³¹ between 03:00 AM and 07:00 AM, was represented by an increase in EGP and a decrease in IS within that time window. Comparing to the training set, the validation set had a 25% increase in the meal bolus at breakfast, a 25% decrease in the basal rate between 8 AM to 4 PM, and a 25% increase in the basal rate between 4 PM and 12 AM. Changes were limited to 25%, taking into account that larger modifications are not generally applied in practice.
Training data were used to solve the optimization problem for h × γ = {2,4,6,8,10} × {0.5,1,10,50,100}, and the validation data were used to compute the Akaike’s Final Prediction Error (FPE):

$$\text{FPE} = \frac{1}{N} {\left(\text{y} - {\text{y}}_{\text{p}}\right)}^{T} \left(\text{y} - {\text{y}}_{\text{p}}\right) \left(\frac{1 + d / N}{1 - d / N}\right)$$

where N is the number of samples, and d is the number of design variables. A fill contour plot of the mean FPE values is presented in FIG. 5. As shown in that figure, the minimum is obtained at h = 8 and γ = {0.5,1}. Since there is no difference in the mean FPE values obtained with these two combinations of h and γ (FPE=291), γ = 1 was selected over γ = 0.5 to further penalize the power of the VC signal.

### Results From Application of the WST According to the UVA Model

### Glucose Reconstruction Using Real Data

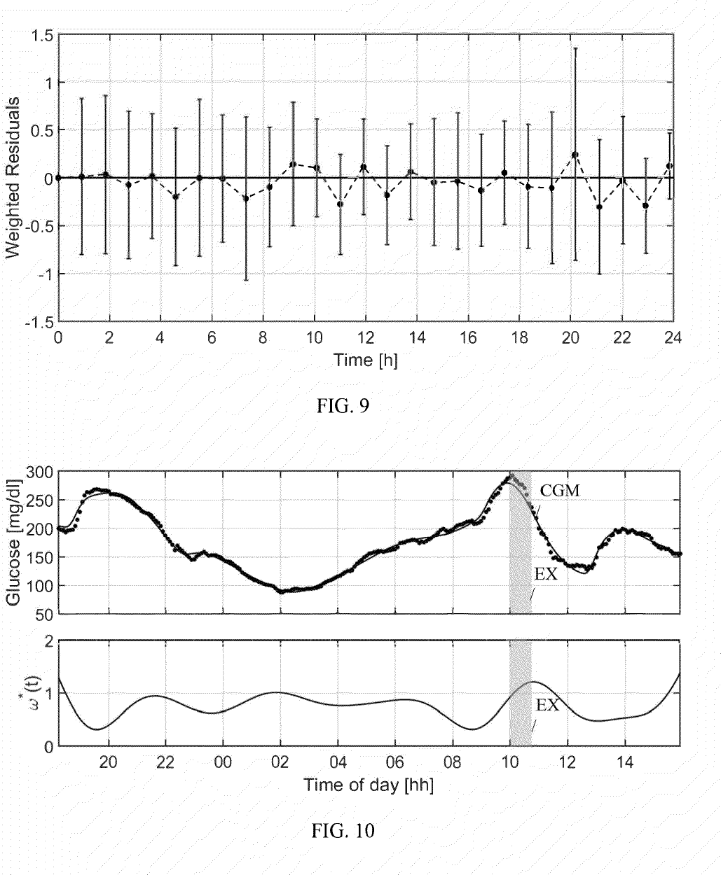

An average root mean squared error (RMSE) of 11.9 ± 5.3 mg/dl was achieved in reconstructing the thirty-eight sets of vectors of real data from subjects with T1D. Average weighted residuals are depicted in FIG. 9. Average estimated parameters (θ^{r}, ϕ) for the population are given in Table 3 below, indicating that they are in agreement with the models’s distribution. For illustrative purposes, an example of glucose reconstruction for one real subject is depicted in FIG. 10 relative to the depicted CGM measurements and exercise period EX.

TABLE 3

Model: A priori distribution

| Parameter | Mean (SD) | Parameter | Mean (SD) |
|---|---|---|---|
| CL | 1.176 (0.339) | Vₘₓ | 0.073 (0.026) |
| k₁ | 0.081 (0.017) | EGP_{b} | 2.482 (0.338) |
| Kₘ₀ | 225.163 (20.377) | k₂ | 0.115 (0.052) |
| kₚ₂ | 0.005 (0.004) | V_{g} | 1.838 (0.133) |
| CL | 1.021 (0.308) | Vₘₓ | 0.081 (0.033) |
| k₁ | 0.081 (0.023) | EGP_{b} | 2.504 (0.391) |
| Kₘ₀ | 224.281 (12.264) | k₂ | 0.137 (0.053) |
| kₚ₂ | 0.005 (0.004) | V_{g} | 1.870 (0.102) |
| ϕ₁ | 0.880 (0.178) | ϕ₂ | 0.143 (0.289) |
| ϕ₃ | -0.002 (0.244) | ϕ₄ | 0.090 (0.189) |
| ϕ₅ | -0.076 (0.241) | ϕ₆ | 0.043 (0.186) |
| ϕ₇ | 0.013 (0.240) | ϕ₈ | 0.138 (0.173) |
| ϕ₉ | 0.030 (0.268) | ϕ₁₀ | 0.073 (0.196) |
| ϕ₁₁ | -0.083 (0.199) | ϕ₁₂ | -0.009 (0.174) |
| ϕ₁₃ | -0.047 (0.188) | ϕ₁₄ | -0.023 (0.138) |
| ϕ₁₅ | -0.034 (0.161) | ϕ₁₆ | -0.032 (0.142) |
| ϕ₁₇ | -0.016 (0.133) | | |

### Glucose Prediction Using Synthetic Data

Several testing sets were generated to substantiate the ability of the proposed methodology in predicting glucose traces under basal rate and meal bolus adjustments. The RMSE was computed to mathematically quantify the error in the estimation, and error grid analysis (EGA) was used for clinical interpretation of the results. In the EGA, glucose estimates are plotted against reference values, and categorized into five accuracy zones. Values in zones A and B are clinically acceptable, whereas values in zones C (risk of unnecessary corrections), D (dangerous failure to detect hypo- or hyperglycemia), and E (erroneous treatment) represent clinically significant errors.

### Basal Rate Adjustment

Seven variations of the training set were proposed to test the performance of the replay engine against basal rate adjustment, each one considering an M% change in the basal rate between 8 AM and 4 PM, and a -M% change in the basal rate between 4 PM and 12 AM, with M from -25 to 25 in increments of 5. Average RMSE of glucose predictions across the population monotonically increases from 9.3±3.0 mg/dl (at reconstruction) to 15.3±6.7 mg/dl (25% variation) with a mean MARD value below 10% for all cases (see FIG. 6A). Results from the EGA, as shown in FIG. 6B indicate (with respect to a sampling time of 30 minutes), adjustments of 0% as indicated at “a,” that predictions for the worst-case scenario, i.e., for basal adjustments of 25% as indicated at “b”, fall 91.9% of time into the A-zone, 7.8% into the B-zone, and only 0.3% into the D-zone.

### Meal Bolus Adjustment

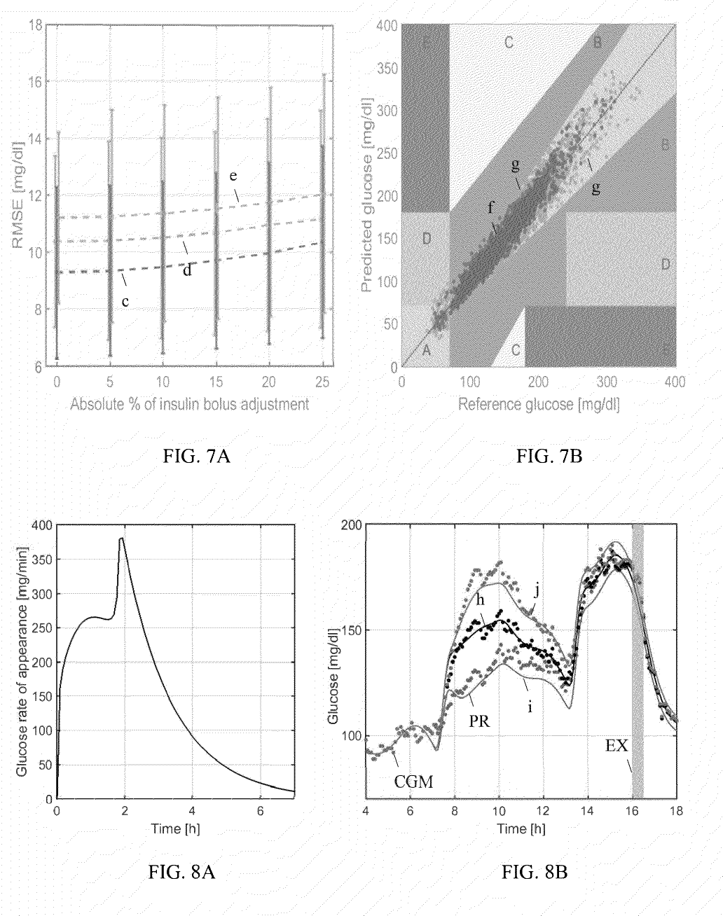

Thirty-three variations of the training set were proposed to test the performance of the replay engine against meal bolus adjustments. Given the meal bolus at breakfast, variations ranging from a 25% decrease to a 25% increase in 5% increments were evaluated under three different conditions: maintaining the original bolus time, and moving it ±30 min. FIGS. 7A and 7B depict the results of this analysis. As expected, the larger the change in the meal bolus, the larger the RMSE of glucose predictions, but maintaining a MARD below 10% for all cases, relative to 0% adjustment at “c,” a forward adjustment of 25% at “d,” and a backward adjustment at “e” (see FIG. 7A). In terms of EGA, predictions fell 97.0% of time into the A-zone, 2.8% into the B-zone, and only 0.2% into the D-zone, relative to the aforementioned respective adjustments indicated at “f” and “g” (see FIG. 7B).

### Mixed-Meals

In order to evaluate how the replay engine might perform under different nutritional compositions and absorption rates, a meal that presents a “double-peak” glucose rate of appearance (see FIG. 8A) was selected from the library reported in Leon-Vargas (2013).³² For illustrative purposes, one in silico subject of the fifty-subject cohort described above was challenged with three mixed meals following the adjuments regime set forth and described for the above meal bolus adjustment. In FIG. 8B, three cases are presented: one case to illustrate the ability of the proposed method to reconstruct the nominal case (no adjustment in the meal bolus at breakfast), and the other two cases to show how the method is able to accurately predict the impact of deviations in both the bolus size and time. Therein, relative to CGM measurements being indicated as such and glucose prediction indicated at “PR” and with exercise represented at EX, indication at “h” signifies no meal bolus adjustment, indication at “i” signifies a 25% increase in the meal bolus and delivery of same 30 minutes before mealtime, and indication at “j” signifies a 25% reduction in the meal bolus and delivery of same 30 minutes after mealtime.

### VC Signal Library of the UVA Model

The collection of VC signals estimated from clinical data was implemented as a library in the time-invariant version of the UVA model to equip the model with a set of inputs that allow testing treatment strategies under more challenging scenarios. At this point, it is worth clarifying that this implementation is an alternative, but not a substitute for the intraday variability introduced in the last version of the model (Visentin et al. (2018).³⁰

To verify that the variability observed in the real data can be reproduced in silico by combining the original time-invariant virtual cohort of the simulator with the proposed library, the following set of simulations for each VC signal ω(t) was performed, considering:
the 100 in silico adults of the simulator without intraday variability, and with default constant insulin basal rates and functional therapy parameters: CR and CF values.
the corresponding real meal input (excluding hypo-treatments).
hypo-treatments and correction boluses administered, on average, as in the clinical trial. For hypo-treatments, a mean rescue frequency, CGM threshold, and size of rescue carbs of 15 min, 76 mg/dl and 11 g, respectively, were estimated. For correction boluses, a mean CGM threshold of 239 mg/dl was observed and a frequency of 1 hour was set.

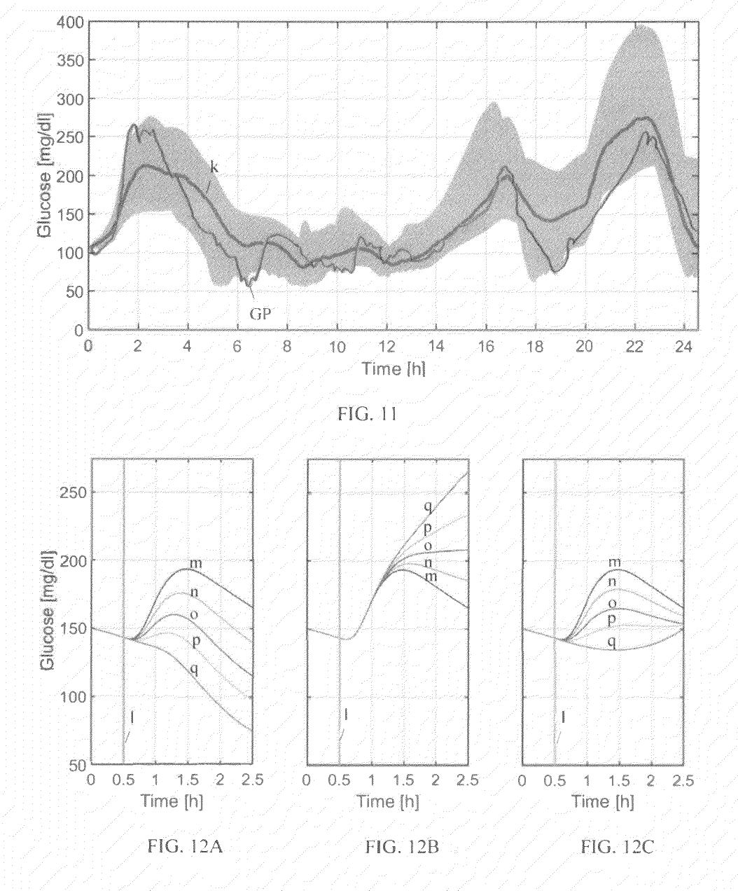

An example showing how the VC signal may be used to equip the UVA model with IS-related variability found in clinical data is provided in FIG. 11, wherein real glucose profile is indicated at “GP,” and line “k” represents the median value from simulated data with boundaries of the filled data signifying the 25% and 75% percentiles. Average results are presented in Table 4 below. Statistical comparisons were determined between test groups using an unpaired two-sample t-test. As shown, no significant difference was detected between simulated and real data.

TABLE 4

| Glucose metric | Real data | Simulated data | p-value |
|---|---|---|---|
| Mean CGM value [mg/dl] | 156.27 (27.69) | 159.58 (13.78) | 0.513 |
| Variability (SD) [mg/dl] | 48.83 (13.92) | 48.87 (10.89) | 0.989 |
| Variability (CV) | 0.31 (0.08) | 0.31 (0.07) | 0.674 |
| % time < 54 mg/dl | 0.31 (1.08) | 0.12 (0.24) | 0.280 |
| % time < 70 mg/dl | 1.99 (2.84) | 1.15 (1.69) | 0.122 |
| % time in [70 140] mg/dl | 42.28 (22.32) | 39.33 (12.59) | 0.480 |
| % time in [70 180] mg/dl | 66.19 (19.82) | 66.55 (11.18) | 0.923 |
| % time > 180 mg/dl | 31.82 (20.38) | 32.3 (11.20) | 0.899 |
| % time > 250 mg/dl | 6.88 (7.65) | 6.2 (4.16) | 0.635 |
| LBGI | 0.67 (0.67) | 0.62 (0.38) | 0.728 |
| HBGI | 6.66 (3.55) | 6.35 (1.98) | 0.642 |

### Adjustment to Meal Size and/or Insulin Bolus

As previously mentioned, the prediction capabilities of the method herein using real data cannot be quantitatively assessed, instead, a qualitative analysis may be performed to show the impact on glucose predictions in response to changing/removing the meal size and insulin bolus at dinner. To this end, the following cases were considered:
(A) varying reductions of the original meal size,
(B) varying reductions of the original insulin bolus, and
(C) varying reductions of both the original meal size and insulin bolus, with variations ranging from 0% to 100% in 25% increments. Average results are illustrated in FIGS. 12A-12C showing median glucose responses to the aforementioned cases (A) through C, respectively. Notably, although predictions depend on the glucose trend at mealtime, the posterior glucose trace remains virtually unaltered when both insulin bolus and meal are removed. In referring to the aforementioned figures, original responses are indicated at “m,” 25% reduction at “n,” 50% reduction at “o,” 75% reduction at “p,” and 100% reduction at “q,” with dinner time being indicated at “1.”

Thus, as may be understood from the above, implementation of the UVA model upon received CGM, meals, and insulin pump data and that incorporates a VC signal capturing variations in IS presents a novel time-varying construction of the T1D virtual image as referred to herein. That is, by providing the VC signal as an insulin variability signal in the form of a truncated Fourier series and estimating θ^{r}, a user of the WST 20 may be enabled to invoke the replay functionality thereof to discern a clinical impact resulting from deviation in aspects regarding meals and insulin doses.

### Studying WST Use

WST 20 was evaluated in a single-arm, uncontrolled, pilot clinical trial of adult subjects at home (ClinicalTrials.gov identifier: NCT04439903). The research protocol was approved by the University of Virginia Institutional Review Board (IRB-HSR 200157). The primary aim of the study was to assess WST’s usability after one month of system use.

Main eligibility criteria included T1D for at least one year, current user of the Tandem t:slim X2™ insulin pump, and willingness to interact with a computer program. Major exclusion criteria included inability to read and complete questionnaires or interact with a computer program, history of a seizure disorder (except hypoglycemia seizure), and pregnancy. Tandem’s t:connect® mobile application was used to consolidate, aggregate, and transmit data automatically from the participants’ insulin pumps to our system’s database. Otherwise, participants would have needed to upload their data to WST manually on a daily basis. This could have affected their attitude towards using the system as most people with T1D using devices download their data very infrequently.²²

Once enrolled, participants attended an interactive training session where they were instructed how to use WST. The first week after training (Phase 1) was purely observational, and data collected during that time was used to estimate the baseline glucose metrics. During the following four weeks (Phase 2), participants were asked to interact with WST at least once a week. Responses to questionnaires were collected pre and post system use.

As a pilot study, this clinical trial was not formally powered to assess effect and therefore, no p-values are reported. Instead, descriptive statistics infer size and direction of treatment effect. The mean and standard deviation (SD) are reported when the distributions of data are normal, and the median and interquartile range [IQR], otherwise.

Fifteen adult participants (four men and eleven women) using Control-IQ® technology completed all study procedures (18 enrollments with 3 screen failures/withdraws). Mean demographics of the study group were as follows: age, 36±13 years; HbA1c, 6.5%±0.7%; weight, 71.2±18.5 kg; and total daily insulin, 35.4±11.8 U.

WST 20 generated models from 86.4% of available days of data, achieving a RMSE of 14.1 mg/dL [9.6 mg/dL,18.3 mg/dL], and a MARD of 6.8% [5.1%,9.1%]. Results from EGA²⁹ indicate that the median percentage of reconstructed glucose values that fell into the clinically acceptable A- and B-zones was 99.7% — A-zone, 95.5% [88.5%,99.0%]; B-zone, 4.2% [1.0%,10.4%]; C-zone, 0.0% [0.0%,0.0%]; D-zone, 0.0% [0.0%,0.4%]; E-zone, 0.0% [0.0%,0.0%].

Mean time using WST per participant was 63.1 min (42.6 min), or approximately 15 min, per participant per week. In terms of interactions with the Display and Replay panels, median numbers of click events on the calendar and Run button were 15 [11,28], and 29 [17,42], respectively.

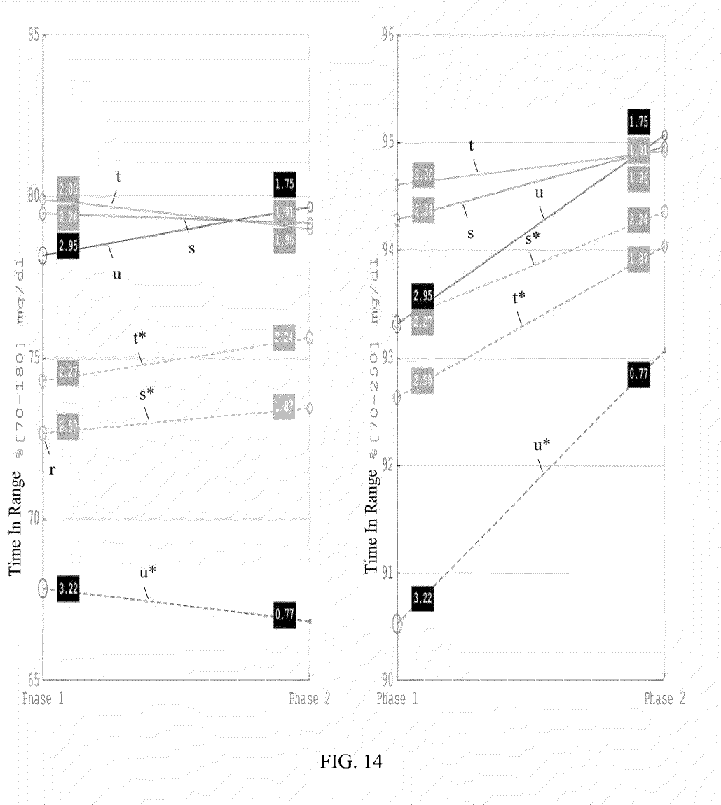

Overall percentage of time in the range [70,180] mg/dL was virtually the same in Phase 1, 79.5% (13.0%), and Phase 2, 79.2% (11.3%), when accounting for one participant exclusion. A slight increase in overall time in the range [70,250] mg/dL was detected (94.3% [91.3%,95.7%] vs 96.2% [92.1%,97.9%]), particularly fueled by a reduction in time below 70 mg/dL (1.6% [0.7%,3.7%] vs 0.8% [0.5%,3.0%]). These changes can be observed especially overnight (time in 70-250 mg/dL: 92.8% [88.3%,98.2%] vs 97.2% [91.8%,99.65]; time < 70 mg/dL: 1.7% [0.0%,4.2%] vs 0.6% [0.0%,2.5%]), and for those participants in this analysis (4) who reported modifying their pumps’ settings based on WST simulations (time in 70-250 mg/dL: 90.0% [88.7%,92.4%] vs 94.5% [87.1%,99.6%]; time < 70 mg/dL: 1.7% [0.0%,6.5%] vs 0.0% [0.0%,0.9%]). Main glycemic outcomes are summarized in Table 5 below, and mean time-in-target results are depicted in FIG. 14. Therein, mean time-in-target, as observed according to the reconstructed glucose time series, is indicated for all participants as to overall time, daytime, and nighttime at “s,” “t,” and “u,” respectively, and for participants who modified their insulin pump settings based on WST 20 replay at “s*,” “t*,” and “u*,” respectively.

TABLE 5

| All participants | Overall | | Overnight | |
|---|---|---|---|---|
| | Phase 1 | Phase 2 | Phase 1 | Phase 2 |
| Mean sensor glucose | 143 (20) | 143 (17) | 140 [121,157] | 133 [120,166] |
| Sensor time <54 mg/dL | 0.4 [0.1,0.7] | 0.1 [0.0,0.7] | 0.2 [0.0,0.8] | 0.0 [0.0,0.0] |
| Sensor time <70 mg/dL | 1.6 [0.7,3.7] | 0.8 [0.5,3.0] | 1.7 [0.0,6.5] | 0.6 [0.0,2.5] |
| Sensor time in [70,180] mg/dL | 79.5 (13.0) | 79.2 (11.3) | 83.2 [67.9,90.5] | 85.4 [65.2,96.9] |
| Sensor time in [70,250] mg/dL | 94.3 [91.3,95.7] | 96.2 [92.1,97.9] | 92.8 [88.3,98.2] | 97.2 [91.8,99.6] |
| Sensor time >180 mg/dL | 18.3 (13.5) | 18.9 (11.3) | 13.9 [1.2,29.0] | 9.9 [2.2,33.8] |
| Sensor time >250 mg/dL | 2.5 [0.3,7.0] | 2.5 [0.2,5.4] | 0.0 [0.0,8.7] | 0.0 [0.0,5.6] |
| Participants who modified their settings based on WST | Overall | | Overnight | |
| | Phase 1 | Phase 2 | Phase 1 | Phase 2 |
| Mean sensor glucose | 153 (18) | 153 (13) | 154 (38) | 162 (30) |
| Sensor time <54 mg/dL | 0.7 [0.5,0.9] | 0.1 [0.0,0.9] | 0.6 [0.0,1.7] | 0.0 [0.0,0.0] |
| Sensor time <70 mg/dL | 2.4 [1.5,3.5] | 0.7 [0.3,3.4] | 1.7 [0.0,6.5] | 0.0 [0.0,0.9] |
| Sensor time in [70,180] mg/dL | 72.7 (11.2) | 73.4 (10.5) | 67.9 (19.5) | 66.8 (22.3) |
| Sensor time in [70,250] mg/dL | 92.6 (1.8) | 94.0 (4.1) | 90.5 (2.7) | 93.1 (6.7) |
| Sensor time >180 mg/dL | 24.8 (12.3) | 24.7 (10.1) | 32.7 [9.4,48.4] | 34.4 [9.0,53.4] |
| Sensor time >250 mg/dL | 4.9 (2.9) | 4.1 (2.8) | 7.2 [2.9,9.6] | 3.0 [0.0,12.3] |

Mean sensor glucose is in mg/dL and sensor time in %.

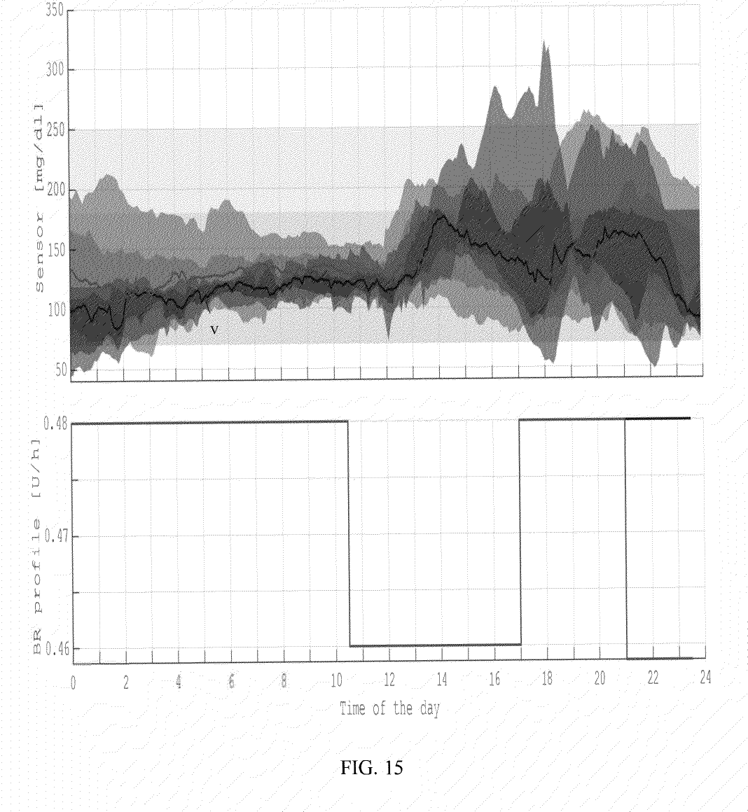

The same conclusions can be drawn from the risks for hypo- and hyperglycemia. Overall Low Blood Glucose Index (LBGI) and High Blood Glucose Index (HBGI)¹⁰ in Phases 1 and 2 were 0.6 [0.3.0.9] vs 0.5 [0.2,0.9] and 4.2 (2.6) vs 4.3 (2.2), respectively. Overnight indices were 0.6 [0.1,1.0] vs 0.4 [0.2,1.0], and 3.6 [1.4,6.1] vs 2.5 [1.1, 6.8], and particularly for the participants who modified their pumps’ settings based on WST simulations were 0.5 [0.0,1.5] vs 0.1 [0.0,0.3], and 6.1 (4.5) vs 6.7 (4.5). An illustrative example from a participant who reported making therapy changes based on WST 20 replay is presented in FIG. 15. As shown, lowering the IBR profile before midnight assisted the participant in reducing the risk for hypoglycemia during nighttime.

In addition to enhancing participants’ better management of their glucose control, as demonstrated above, the study also discerned a decrease in diabetes distress, as measured according to the Diabetes Distress Scale (DDS), a 17-item scale that yields a total diabetes distress score and 4 subscale scores, including emotional burden, regimen distress, interpersonal distress, and physician distress.³² Mean item scores <2.0, between 2.0 and 2.9, and > 3.0, are considered little to no distress, moderate distress, and high distress, respectively. In particular, results showed a reduction in distress level relative to measurements taken before and after system use as emotional burden: 2.5±1.1 vs 2.1±0.8 and regimen distress: 1.6 [1.4,2.5] vs 1.4 [1.2,2.3].

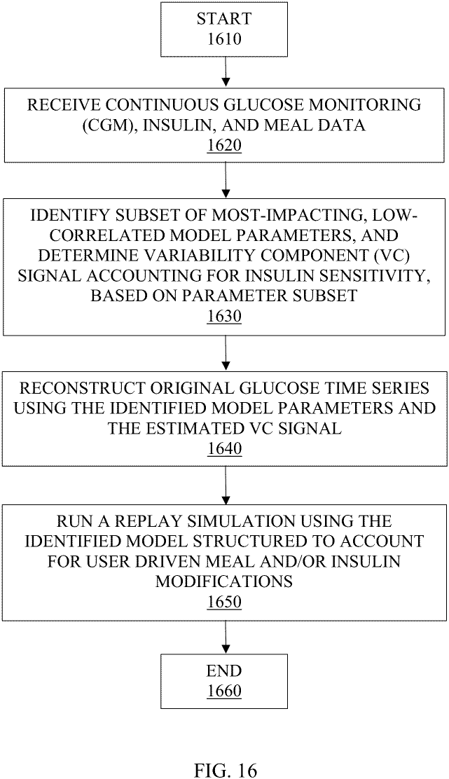

Referring to FIG. 16, which illustrates a high-level block diagram of information processing according to FIGS. 1 and 2, the WST 20 may initiate processing at 1610 and proceed at 1620 to automatically receive CGM, insulin, meal, and therapy profile data from a given user’s insulin pump. From there, the WST 20 may proceed, at 1630, toidentify model parameter subset θ^{r}, and estimate VC signal ω*. Based on such identification and estimation and when feeding back the user’s originally received meal and insulin data into the UVA model, the user’s originally received CGM data may be reconstructed, as at 1640. Given the reconstruction, the WST 20 may then provide the user an opportunity to replay, at 1650, the reconstructed glucose time series so as to account for user driven meal and/or insulin modifications, and thus reflect any resultant change in glucose level. In this way, the user may then be enabled to visualize the modified time series to learn the effect of such modification, and resultingly improve his/her individual diabetes literacy. Collectively, the aforementioned reconstruction and replay have evinced clinically acceptable fitting.

Thus, as may be understood from the above, the WST 20, in implementing the UVA model, effectively enables a user to efficiently realize a reconstruction of their glucose time series as informed by glucose, insulin and meal data automatically received from the user’s insulin pump on a daily basis. As such, the burden of manual user entry is alleviated, while ensuring data accuracy. When attaining the reconstruction, the WST 20 thus obtains a personalized mathematical model of glucose homeostasis that may be used as a testbed for user driven modifications of meal and/or insulin data. This way, the user may, prior to ending WST 20 operations as at 1660, be enabled to reflect upon the impact of such modification(s) when compared to the received data, as reconstructed. Through such reflection, therefore, the user is provided a valuable educational tool according to the WST 20 that endeavors to provide the user with an optimal representation of his/her individual glucose homeostasis. In providing such tool, the WST 20, through its implementation of the UVA model as discussed herein, presents a significant and practical application of such modeling of a glucose time series as derived firstly from automated receipt of a patient’s glucose, meal, and insulin data and secondly from user driven modifications to a reconstruction for such time series. As such, the modeling and its results may be invoked to counteract combined disadvantages of prior systems, including potential for inaccuracy in glucose analysis resulting from manual entry of initial data therefor and an inability of a patient to individually modify modeled data to learn an effect of modification thereon.

Although the present embodiments have been described in detail, those skilled in the art will understand that various changes, substitutions, variations, enhancements, nuances, gradations, lesser forms, alterations, revisions, improvements and knock-offs of the embodiments disclosed herein may be made without departing from the spirit and scope of the embodiments in their broadest form.

Where applicable, citations herein, whether by numerical indication or by other means, refer to one or more of the documents listed in the section entitled “References.”

### REFERENCES

The devices, systems, apparatuses, compositions, computer program products, non-transitory computer readable medium, models, algorithms, and methods of various embodiments disclosed herein may utilize aspects (e.g., devices, systems, apparatuses, compositions, computer program products, non-transitory computer readable medium, models, algorithms, and methods) disclosed in the following references, applications, publications and patents and which are hereby incorporated by reference herein in their entirety, and which are not admitted to be prior art with respect to embodiments herein by inclusion in this section:

1. Diabetes Control and Complications Trial Research Group. The effect of intensive treatment of diabetes on the development and progression of long-term complications in insulin-dependent diabetes mellitus. N Engl J Med. 1993;329(14):977-986. doi: 10.1056/NEJM199309303291401.

2. Nathan DM, DCCT/EDIC Research Group. The diabetes control and complications trial/epidemiology of diabetes interventions and complications study at 30 years:

Overview. Diabetes Care. 2014;37(1):9-16. doi:10.2337/dc13-2112.

3. Nathan DM, Cleary PA, Backlund J-YC, et al. Intensive diabetes treatment and cardiovascular disease in patients with type 1 diabetes. N Engl J Med. 2005;353(25):2643-2653. doi:10.1056/nejmoa052187.

4. Miller KM, Foster NC, Beck RW, et al. Current state of type 1 diabetes treatment in the U.S.: Updated data from the T1D exchange clinic registry. Diabetes Care. 2015;38(6):971-978. doi:10.2337/dc15-0078.

5. Fitzsimons B, Wilton L, Lamont T, McCulloch L, Boyce J. The Audit Commission review of diabetes services in England and Wales, 1998-2001. Diabet Med. 2002;19(SUPPL. 4):73-78. doi:10.1046/j.1464-5491.19.s4.13.x.

6. Etzwiler DD. Diabetes translation: A blueprint for the future. Diabetes Care. 1994;17(SUPPL. 1):1-4.

7. Waldhäusl WK. The physiological basis of insulin treatment - clinical aspects. Diabetologia. 1986;29(12):837-849. doi:10.1007/BF00870138.

8. O’Malley G, Messer LH, Levy CJ, et al. Clinical management and pump parameter adjustment of the Control-IQ closed-loop control system: Results from a 6-month, multicenter, randomized clinical trial. Diabetes Technol Ther. 2021;23(4):245-252. doi:10.1089/dia.2020.0472.

9. Reddy M, Rilstone S, Cooper P, Oliver NS. Type 1 diabetes in adults: Supporting self management. BMJ. 2016;352:i998. doi:10.1136/bmj.i998.

10. Kovatchev BP. Metrics for glycaemic control-from HbA1c to continuous glucose monitoring. Nat Rev Endocrinol. 2017;13(7):425-436. doi:10.1038/nrendo.2017.3.

11. Kim BYB, Lee J. Smart devices for older adults managing chronic disease: A scoping review. JMIR mHealth uHealth. 2017;5(5). doi:10.2196/mhealth.7141.

12. Singh K, Drouin K, Newmark LP, et al. Patient-facing mobile apps to treat high-need, high-cost populations: A scoping review. JMIR mHealth uHealth. 2016;4(4):e6445. doi:10.2196/mhealth.6445.

13. Kaufman N, Khurana I. Using digital health technology to prevent and treat diabetes. Diabetes Technol Ther. 2016;18(Suppl 1):S56-S68. doi:10.1089/dia.2016.2506.

14. Tyler NS, Jacobs PG. Artificial intelligence in decision support systems for type 1 diabetes. Sensors (Switzerland). 2020;20(11). doi:10.3390/s20113214.

15. Lehmann ED. Interactive Educational Diabetes Simulators: A Look to the Future. Vol 2. Mary Ann Liebert, Inc; 2000. www.liebertpub.com. Accessed May 4, 2021.

16. Salzsieder E, Vogt L, Kohnert KD, Heinke P, Augstein P. Model-based decision support in diabetes care. Comput Methods Programs Biomed. 2011;102(2):206-218. doi:10.1016/j.cmpb.2010.06.001.

17. Cavan DA, Everett J, Plougmann S, Hejlesen OK. Use of the Internet to optimize self-management of type 1 diabetes: Preliminary experience with DiasNet. J Telemed Telecare. 2003;9(Suppl 1):50-52. doi:10.1258/135763303322196330.

18. Sperl-Hillen JA, O’Connor P, Ekstrom H, et al. Using simulation technology to teach diabetes care management skills to resident physicians. J Diabetes Sci Technol. 2013;7(5):1243-1254. doi:10.1177/193229681300700514.

19. Hejlesen OK, Andreassen S, Frandsen NE, et al. Using a double blind controlled clinical trial to evaluate the function of a Diabetes Advisory System: A feasible approach? Comput Methods Programs Biomed. 1998;56(2):165-173. doi:10.1016/S0169-2607(98)00023-6.

20. Dinesen B, Andersen PER. Qualitative evaluation of a diabetes advisory system, DiasNet. J Telemed Telecare. 2006;12(2):71-74. doi:10.1258/135763306776084329.

21. Colmegna P, Wang K, Garcia-Tirado J, Breton MD. Mapping data to virtual patients in type 1 diabetes. Control Eng Pract. 2020;103:104605. doi:10.1016/j.conengprac.2020.104605.

22. Beck RW. Downloading diabetes device data: Empowering patients to download at home to achieve better outcomes. Diabetes Technol Ther. 2015;17(8):536-537. doi:10.1089/dia.2015.0169.

23. Weissberg-Benchell J, Hessler D, Polonsky WH, Fisher L. Psychosocial impact of the Bionic Pancreas during summer camp. J Diabetes Sci Technol. 2016;10(4):840-844. doi:10.1177/1932296816640289.

24. Polonsky WH, Fisher L, Earles J, et al. Assessing psychosocial distress in diabetes: Development of the Diabetes Distress Scale. Diabetes Care. 2005;28(3):626-631. doi:10.2337/diacare.28.3.626.

25. Clarke WL. The original Clarke Error Grid Analysis (EGA). Diabetes Technol Ther. 2005;7(5):776-779.

26. Shrivastava SRBL, Shrivastava PS, Ramasamy J. Role of self-care in management of diabetes mellitus. J Diabetes Metab Disord. 2013;12(14):1-5. doi:10.1186/2251-6581-12-14.

27. Plougmann S, Hejlesen OK, Cavan DA. DiasNet - A diabetes advisory system for communication and education via the internet. Int J Med Inform. 2001;64(2-3):319-330. doi:10.1016/S1386-5056(01)00214-3.

28. Brun, R., Reichert, P., & Kunsch, H.R. (2001), Practical identifiability analysis of large environmental simulation models. Water Resources Research, 37, 1015-1030. doi: 10. 1029/2000WR900350.

29. Visentin, R., Dalla Mann, C., Kudva, Y., Basu, A., & Cobelli, C. (2015). Circadian variability of insulin sensitivity; Physiological input for in silico artifical pancreas. Diabetes Technol Ther, 17, 1-7.

30. Visentin, R., Campos-Nunez, E., Schiavon, M., Lv, D., Vettoretti, M., Breton, M. et al. (2018). The UVA/Padova type 1 diabetes simulator goes from single meal to single day. J Diabetes Sci Technol, 17, 273-281.

31. Mallard, A., Hinshaw, L., Dalla Man, C., Cobelli, C., Basu, R., Lingineni, R. et al. (2015). Nocturnal glucose metabolism in type 1 diabetes: A study comparing single versus dual tracer approaches. Diabetes Technol Ther, 17, 587-595.

32. Leon-Vargas, F. (2013). Design and implementation of a closed-loop blood glucose control system in patients with type 1 diabetes. Ph.D. thesis. Universitat de Girona.

33. Ramkisson, C. M., Bertachi, A. Beneyto, A., Bondia, J. & Vehi, J. (2020). Detection and control of unannounced exercise in the artificial pancreas without additional physiological signals. IEEE J BiomedHealth, 24, 259-267.

34. Goodwin, G. C., Seron, M. M., Medioli, A. M., Smith, T., King, B. R., & Smart, C. E. (2020). A systematic stochastic design strategy achieving an optimal tradeoff between peak BGL and probability of hypoglycaemic events for individuals having type 1 diabetes melititus. Biomed Signal Process Control, 57, 101813.

35. Dalla Man, C., Micheletto, F., Lv, D., Breton, M., Kovatchev, B., & Cobelli, C. (2014). The UVA/Padova type 1 diabetes simulator: New features. J Diabetes Sci Technol, 8, 26-34.

What is claimed is:

1. A processor-implemented method for modeling a time-varying representation of the glucose homeostasis of a patient with Type 1 diabetes (T1D) according to a computational model driven by the processor, said method comprising:
retrieving from a storage a dataset for said patient comprising continuous glucose monitoring (CGM), insulin, and meal records collected from one or more devices associated with said patient, said dataset being automatedly deposited into said storage at one or more predetermined time intervals;
determining, according to operation of said model on said dataset, a subset (θ^{r}) of most-impacting, low-correlated model parameters, along with a variability control (VC) signal accounting for insulin sensitivity (IS) of said patient;
formulating, based on said model being informed by each of said θ^{r}, VC signal and dataset, a reconstructed glucose time series for said patient;
introducing to said model patient provided modification of one or more of said insulin and meal records; and
based on said modification, generating by said model a replay of said reconstructed glucose time series for said patient that reflects an effect of said modification.

2. The method of claim 1, wherein:
providing a user interface interface operably coupled to said processor to receive said modification.

3. The method of claim 2, wherein:
said VC signal comprises a truncated Fourier series capturing daily variation in IS.

4. The method of claim 3, wherein:
said VC signal comprises a number of harmonics of said truncated Fourier series and a predetermined magnitude of a tuning parameter selected to penalize a power of said VC signal.

5. The method of claim 4, wherein:
said VC signal modulates an impact of insulin on endogenous glucose production and insulin-dependent glucose utilization when generating, respectively, said reconstructed glucose time series and said replay thereof.

6. The method of claim 5, wherein:
θ^{r} comprises at least insulin clearance (CL), distribution volume of glucose (V_{g}), a first diffusion constant of said model (k₁), basal endogenous glucose production (EGP_{b}), a second diffusion constant of said model (k₂), and liver glucose effectiveness (kₚ₂).

7. A system for modeling a time-varying representation of the glucose homeostasis of a patient with Type 1 diabetes (T1D) according to a computational model, comprising:
a processor;
a processor-readable memory comprising processor-executable instructions for:
retrieving from a storage a dataset for said patient comprising continuous glucose monitoring (CGM), insulin, and meal records collected from one or more devices associated with said patient, said dataset being automatedly deposited into said storage at one or more predetermined time intervals;
determining, according to operation of said model on said dataset, a subset (θ^{r}) of most-impacting, low-correlated model parameters, along with a variability control (VC) signal accounting for insulin sensitivity (IS) of said patient;
formulating, based on said model being informed by each of said θ^{r}, VC signal and dataset, a reconstructed glucose time series for said patient;
introducing to said model patient provided modification of one or more of said insulin and meal records; and
based on said modification, generating by said model a replay of said reconstructed glucose time series for said patient that reflects an effect of said modification.

8. The system of claim 7, further comprising:
a user interface operably coupled to said processor to receive said modification.

9. The system of claim 8, wherein:
said VC signal comprises a truncated Fourier series capturing daily variation in IS.

10. The system of claim 9, wherein:
said VC signal comprises a number of harmonics of said truncated Fourier series and a predetermined magnitude of a tuning parameter selected to penalize a power of said VC signal.

11. The system of claim 10, wherein:
said VC signal modulates an impact of insulin on endogenous glucose production and insulin-dependent glucose utilization when generating, respectively, said reconstructed glucose time series and said replay thereof.

12. The system of claim 11, wherein:
θ^{r} comprises at least insulin clearance (CL), distribution volume of glucose (V_{g}), a first diffusion constant of said model (k₁), basal endogenous glucose production (EGP_{b}), a second diffusion constant of said model (k₂), and liver glucose effectiveness (kₚ₂).

13. A non-transient computer-readable medium having stored thereon computer-executable instructions for modeling a time-varying representation of the glucose homeostasis of a patient with Type 1 diabetes (T1D) according to a computational model, said instructions comprising instructions causing a computer to:
retrieve from a storage a dataset for said patient comprising continuous glucose monitoring (CGM), insulin, and meal records collected from one or more devices associated with said patient, said dataset being automatedly deposited into said storage at one or more predetermined time intervals;
determine, according to operation of said model on said dataset, a subset (θ^{r}) of most-impacting, low-correlated model parameters, along with a variability control (VC) signal accounting for insulin sensitivity (IS) of said patient;
formulate, based on said model being informed by each of said θ^{r}, VC signal and dataset, a reconstructed glucose time series for said patient;
introduce to said model patient provided modification of one or more of said insulin and meal records; and
based on said modification, generate by said model a replay of said reconstructed glucose time series for said patient that reflects an effect of said modification.

14. The computer-readable medium of claim 13, wherein:
said modification is configured for said introduction via a user interface configured to be operably coupled to said computer.

15. The computer-readable medium of claim 14, wherein:
said VC signal comprises a truncated Fourier series capturing daily variation in IS.

16. The computer-readable medium of claim 15, wherein:
said VC signal comprises a number of harmonics of said truncated Fourier series and a predetermined magnitude of a tuning parameter selected to penalize a power of said VC signal.

17. The computer-readable medium of claim 16, wherein:
said VC signal modulates an impact of insulin on endogenous glucose production and insulin-dependent glucose utilization when generating, respectively, said reconstructed glucose time series and said replay thereof.

18. The computer-readable medium of claim 17, wherein:
θ^{r} comprises at least insulin clearance (CL), distribution volume of glucose (V_{g}), a first diffusion constant of said model (k₁), basal endogenous glucose production (EGP_{b}), second kinetics (Kₘ₀), a second diffusion constant of said model (k₂), and liver glucose effectiveness (kₚ₂).
